(19) United States
(12) Patent Application Publication
(10) Pub. No.: US 2024/0327783 A1
(43) Pub. Date: Oct. 3, 2024

(54) ROOM-TEMPERATURE-STABLE CULTURE MEDIA AND SUPPLEMENTS

(71) Applicant: Life Technologies Corporation, Carlsbad, CA (US)

(72) Inventors: Joseph White, Williamsville, NY (US); Matthew Smonskey, Orchard Park, NY (US); Thomas Mellody, Snyder, NY (US)

(21) Appl. No.: 18/435,956

(22) Filed: Feb. 7, 2024

Related U.S. Application Data
(60) Provisional application No. 63/443,768, filed on Feb. 7, 2023.

(51) Int. Cl. C12N 5/00; C12N 5/073

(52) U.S. Cl. CPC C12N 5/0018; C12N 5/0603; C12N 5/0604; C12N 2500/32; C12N 2500/34; C12N 2500/36; C12N 2500/38

(57) ABSTRACT

Described herein are formulations of cell culture media and supplements that are stable when stored at room temperature. Embodiments described herein encompass dry granulated or agglomerated cell culture media with carbohydrate sources and vitamins that are physically separated. Further embodiments described herein are dry granulated or agglomerated cell culture media with a carbohydrate source that is separately agglomerated or powdered. Additional embodiments are dry granulated or agglomerated cell culture media with a carbohydrate source that is separately agglomerated or powdered and optionally contains vitamins. In another embodiment, the culture media or supplement is a dry granulated or agglomerated cell culture media containing granulated or agglomerated or powdered carbohydrates and vitamins in one or more of a liquid-, powdered-, granulated-, agglomerated-, coated-, or encapsulated-form.

### CROSS-REFERENCE TO RELATED APPLICATION

This application claims the benefit under 35 U.S.C. § 119(e) of U.S. Provisional Patent Application No. 63/443,768 filed Feb. 7, 2023. The entire contents of the application is incorporated herein by reference.

### TECHNICAL FIELD

Described herein are formulations of cell culture media and supplements that are stable when stored at room temperature. Embodiments described herein encompass dry granulated or agglomerated cell culture media with carbohydrate sources and vitamins that are physically separated. Further embodiments described herein are dry granulated or agglomerated cell culture media with a carbohydrate source that is separately agglomerated or powdered. Additional embodiments are dry granulated or agglomerated cell culture media with a carbohydrate source that is separately agglomerated or powdered and optionally contains vitamins. In another embodiment, the culture media or supplement is a dry granulated or agglomerated cell culture media containing granulated or agglomerated or powdered carbohydrates and vitamins in one or more of a liquid-, powdered-, granulated-, agglomerated-, coated-, or encapsulated-form.

### BACKGROUND

Cell culture media provide the nutrients necessary to maintain and grow cells in a controlled, artificial, and in vitro environment. Characteristics and compositions of the cell culture media vary depending on the particular cellular requirements. Important parameters include osmolality, pH, and nutrient formulations.

A typical cell culture media contains ions (e.g., sodium, potassium, calcium, magnesium, chloride, phosphate, sulfate), amino acids, vitamins, trace elements, and sugar sources. Buffers, such as sodium bicarbonate, antibiotics, and a pH indicator (e.g., phenol red) are often added. Serum, such as fetal bovine serum (FBS), is typically added to augment cell growth. FBS often contains more than 1000 components, including proteins, electrolytes, lipids, carbohydrates, hormones, enzymes, and other undefined constituents. Advanced granulation technology (AGT) revolutionized media by providing media in an agglomerated format that contains basal media and serum in a format that dissolves easily and does not create potentially hazardous dust.

One issue with prepared agglomerated complete media formulations that contain sugar sources (e.g., glucose) and vitamins and other labile nutrients is that they typically require refrigerated storage to prevent degradation of media components that are important for cell culture stability. Amino acids are essential nutrients for cell growth and viability. At ambient temperatures, the carbohydrate sources can engage in Maillard reactions with other components, such as amino acids, and degrade these components. The mechanisms behind the Maillard reaction have been known since the early to mid-1900's. However, it's negative effects on cell culture medium have only recently been investigated. In fact, Maillard reaction products (MRPs) affect cell culture stability by depleting amino acids and peptides that react with carbonyl groups on reducing sugars. Vitamins in cell culture medium also degrade during room-temperature storage. Thus, certain vitamins are subject to the Maillard reaction. For example, ascorbic acid can be destroyed by heat. It is known that ascorbic acid and its degradation products participate in chemical modifications of proteins in vivo through the Maillard reaction. Another vitamin that can be degraded by the Maillard reaction is para-amino benzoic acid (PABA) which has been shown to degrade even at refrigerated temperatures over many months.

MRPs are well known in the food industry where the Maillard reaction that occurs in the presence of heat between amino acids and reducing sugars results in food browning and thereby produces fresh aromas and flavors. Depending on the way the food is processed, both beneficial and toxic MRPs can be produced. In cell culture, less is known about the effects of MRPs and what can be done to reduce potentially harmful effects. The challenge with cell culture media stored at room temperature is that the ambient temperature can increase deplete nutrients and increase toxic MRPs. What is needed are cell culture media and supplement formulations that are stable at room temperature and are convenient for large scale use.

### SUMMARY

One embodiment described herein is a room-temperature-stable cell culture media or supplement composition comprising: a base cell culture medium comprising one or more amino acids, salts, buffers, trace minerals, lipids, nucleic acids, or proteins; one or more carbohydrates; and one or more vitamins; wherein the carbohydrates and vitamins are segregated from the base cell culture medium. In one aspect, the composition is stable at a temperature from about 15° C. to about 30° C. In another aspect, the composition is stable at room temperature for about 1 day to about 2 years. In another aspect, the segregation comprises one or more of: physical separation of the base cell culture medium from the carbohydrates and/or vitamins in different containers; separation of the base cell culture medium from the carbohydrates and/or vitamins in the same container; granulation or agglomeration of the base cell culture medium; powdering, granulation, or agglomeration of the carbohydrates; powdering, granulation, agglomeration, encapsulation, or coating of the vitamins; or combinations thereof. In another aspect, the carbohydrates and/or vitamins are intermixed with the base cell culture medium to form the temperature-stable cell culture media or supplement composition. In another aspect, the base cell culture medium is granulated or agglomerated and situated in a first container; the carbohydrates are granulated or agglomerated and situated in a second container; and the vitamins are situated in a third container. In another aspect, the base cell culture medium is granulated or agglomerated and situated in a first container; the carbohydrates are powdered and situated in a second container; and the vitamins are situated in a third container; the vitamins may be agglomerated, powdered, or in liquid form. In another aspect, the base cell culture medium and the carbohydrates are separately granulated or agglomerated, admixed, and situated in a first container; and the vitamins are situated in a second container; the vitamins may be agglomerated, powdered, or in liquid form. In another aspect, the base cell culture medium is granulated or agglomerated, the carbohydrates are powdered and admixed with the agglomerated base cell culture medium and situated in a first container; and the vitamins are situated in a second container; the vitamins may be agglomerated, powdered, or in liquid form. In another aspect, the base cell culture medium is granulated or agglomerated and situated in a first container; and the carbohydrates and the vitamins are admixed, powdered, granulated, or agglomerated, and situated in a second container. In one aspect, the carbohydrates and/or the vitamins are powdered or agglomerated, or a combination thereof. In another aspect, the base cell culture medium is granulated or agglomerated and situated in a first container; the carbohydrates are granulated or agglomerated, admixed with the granulated or agglomerated base cell culture medium and situated in the first container; the vitamins are situated in a third container; the vitamins may be agglomerated, powdered, or in liquid form. In another aspect, the base cell culture medium is granulated or agglomerated and situated in a first container; the carbohydrates are powdered, admixed with the granulated or agglomerated base cell culture medium and situated in the first container; the vitamins are situated in a third container; the vitamins may be powered, agglomerated, or in liquid form. In another aspect, the interaction or chemical reaction of the carbohydrates and/or vitamins with the base cell culture medium is inhibited or reduced owing to segregation in separate containers. In another aspect, the carbohydrates comprise one or more of glucose, fructose, or trehalose. In another aspect, the one or more vitamins comprise one or more of retinol (A), thiamine (B1), riboflavin (B2), niacinamide (B3), pantothenic acid (B5), pyridoxamine (B6), biotin (B7), folic acid (B9) cobalamin (B12), ascorbic acid (C), cholecalciferol (D), tocopherol (E), phylloquinone (K), choline, inositol, lipoic acid, or para-aminobenzoic acid.

Another embodiment described herein is a room-temperature-stable cell culture media composition comprising: granules or agglomerates of a base cell culture medium comprising one or more amino acids, salts, buffers, trace minerals, lipids, nucleic acids, or proteins; and granules or agglomerates of one or more carbohydrates. In one aspect, the granules or agglomerates of the base cell culture medium are admixed with the granules or agglomerates of one or more carbohydrates. In another aspect, the granules or agglomerates of the base cell culture medium are layered over the granules or agglomerates of one or more carbohydrates; or the granules or agglomerates of one or more carbohydrates are layered over the granules or agglomerates of the base cell culture medium.

Another embodiment described herein is a room-temperature-stable cell culture media composition comprising granules or agglomerates of one or more carbohydrates and one or more vitamins.

Another embodiment described herein is a room-temperature-stable cell culture media composition comprising: granules or agglomerates of a base cell culture medium comprising one or more amino acids, salts, buffers, trace minerals, lipids, nucleic acids, or proteins; and granules or agglomerates of one or more carbohydrates and one or more vitamins. In one aspect, the granules or agglomerates of the base cell culture medium are admixed with the granules or agglomerates of one or more carbohydrates and one or more vitamins. In another aspect, the granules or agglomerates of the base cell culture medium are layered over the granules or agglomerates of one or more carbohydrates and one or more vitamins; or the granules or agglomerates of one or more carbohydrates and one or more vitamins are layered over the granules or agglomerates of the base cell culture medium.

Another embodiment described herein is a room-temperature-stable cell culture media composition comprising powders of one or more carbohydrates and one or more vitamins.

Another embodiment described herein is a room-temperature-stable cell culture media composition comprising: granules or agglomerates of a base cell culture medium comprising one or more amino acids, salts, buffers, trace minerals, lipids, nucleic acids, or proteins; and powders of one or more carbohydrates and one or more vitamins. In one aspect, the granules or agglomerates of the base cell culture medium are admixed with the powders of one or more carbohydrates and one or more vitamins. In another aspect, the granules or agglomerates of the base cell culture medium are layered over the powders of one or more carbohydrates and one or more vitamins; or the powders of one or more carbohydrates and one or more vitamins are layered over the granules or agglomerates of the base cell culture medium.

Another embodiment described herein is a room-temperature-stable cell culture media composition comprising: granules or agglomerates of a base cell culture medium comprising one or more amino acids, salts, buffers, trace minerals, lipids, nucleic acids, or proteins; and granules or agglomerates of one or more carbohydrates; and one or more encapsulated or coated vitamins. In one aspect, the granules or agglomerates of the base cell culture medium are admixed with the granules or agglomerates of one or more carbohydrates; and the encapsulated vitamins are admixed with the admixture of the base cell culture medium and carbohydrates or layer over or under the admixture of the base cell culture medium and carbohydrates. In another aspect, the vitamins are coated or incapsulated in one or more of alginate, agar, agarose, pectin, or guar gum.

Another embodiment described herein is a method of manufacturing the room-temperature-stable cell culture composition described herein, the method comprising: (a) combining the one or more amino acids, salts, buffers, trace minerals, lipids, nucleic acids, or proteins to form the base cell culture medium; (b) granulating or agglomerating the base cell culture medium; (c) combining the granulated or agglomerated the base cell culture medium with carbohydrates. In one aspect, the method further comprises preparing a solution concentrate of one or more vitamins. In another aspect, the method further comprises adding the vitamin solution concentrate to the combination of the base cell culture medium and carbohydrates upon solubilization. In another aspect, steps (c)-(d) comprises: (c) granulating or agglomerating carbohydrates and vitamins; and (d) combining the granulated or agglomerated base cell culture medium with the granulated or agglomerated carbohydrates and vitamins. In another aspect, steps (c)-(d) comprises: (c)(i) granulating or agglomerating the carbohydrates; and (c)(ii) encapsulating or coating the vitamins; (d) combining the granulated or agglomerated base cell culture medium with the granulated or agglomerated carbohydrates and the coated or encapsulated vitamins.

Another embodiment described herein is a method for making a liquid cell culture media, the method comprising solubilizing any of the compositions described herein with water.

In one aspect, the method further comprises contacting one or more mammalian cells with the liquid media and culturing the cells.

Another embodiment described herein is a kit for storing a room temperature-stable cell culture media comprising: (a) a base cell culture medium comprising one or more amino acids, salts, buffers, trace minerals, lipids, nucleic acids, or proteins, wherein the base cell culture medium is optionally granulated or agglomerated; (b) one or more carbohydrates, wherein the carbohydrates are optionally granulated or agglomerated; (c) one or more vitamins; wherein the vitamins are optionally granulated, agglomerated, encapsulated, coated, powdered, or in a solution concentrate; and (d) one or more containers comprising drums, buckets, boxes, bags, pouches, or combinations thereof. In one aspect, the one or more carbohydrates are powdered. In another aspect, the base cell culture medium comprises granules or agglomerates situated in a first container; the carbohydrates comprise separate granules or agglomerates situated in a second container; and the vitamins comprise a solution concentrate situated in a third container. In another aspect, the base cell culture medium comprises granules or agglomerates, and the carbohydrates comprise separate granules or agglomerates, both of which are mixed and situated in a first container; and the vitamins comprise a solution concentrate situated in a second container. In another aspect, the base cell culture medium comprises granules or agglomerates, the carbohydrates and vitamins are combined in mixed granules or agglomerates, each of which are present as an intermixed composition situated within a single container. In another aspect, the base cell culture medium comprises granules or agglomerates, and the carbohydrates comprise separate granules or agglomerates, both of which are situated in a first container, wherein the cell culture medium granules or agglomerates is layered over the carbohydrate granules or agglomerates or the carbohydrates granules or agglomerates is layered over the cell culture medium granules or agglomerates; and the vitamins comprise a solution concentrate situated in a second container. In another aspect, the base cell culture medium comprises granules or agglomerates; the carbohydrates and vitamins are combined in mixed granules or agglomerates; each of which are present as an intermixed composition situated within a single container. In another aspect, the base cell culture medium comprises granules or agglomerates; the carbohydrates comprise separate granules or agglomerates; and the vitamins comprise encapsulated particles; each of which are present as an intermixed composition situated within a single container. In another aspect, the interaction or chemical reaction of the carbohydrates and/or vitamins with the base cell culture medium is inhibited or reduced. In another aspect, the kit optionally further comprises one or more of labels, manufacturing dates, expiration dates, use by dates, packaging, branding, instructions for use, material safety data sheets (MSDS), hazard or warning placards, or product literature.

Another embodiment described herein is a method of making a cell culture media using any of the kits described herein, the method comprises combining the kit components (a)-(c) and solubilizing the components with water, forming a liquid medium. In one aspect, the method further comprises contacting one or more mammalian cells with the liquid medium and culturing the cells.

Another embodiment described herein is the use of any of the compositions or the kits for cell culture media. In one aspect, the media is used for culturing a mammalian cell in vitro. In another aspect, the mammalian cell comprises CHO cells or HEK cells.

Other features and advantages of the disclosure will be apparent from the following detailed description and from the claims.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-F show exemplary illustrations of the media formulations and kits described herein. FIG. 1A shows a media kit comprising separate containers comprising agglomerated bulk media, agglomerated or powdered carbohydrate (e.g., glucose), and a 100× vitamin concentrate. FIG. 1B shows a media kit comprising a mixture of an agglomerated bulk media with agglomerated or powdered carbohydrate (e.g., glucose) in one container and a 100× vitamin concentrate in a separate container. FIG. 1C shows a media kit comprising agglomerated bulk media in one container and agglomerated or powdered carbohydrate (e.g., glucose) and vitamins in a separate container. FIG. 1D shows a media kit comprising an agglomerated bulk media layered over agglomerated or powdered carbohydrate (e.g., glucose) in one container and a 100× vitamin concentrate in a separate container. The agglomerated or powdered carbohydrate could be layered over the agglomerated bulk media in a different aspect. FIG. 1E shows a media kit comprising a mixture of an agglomerated bulk media with agglomerated or powdered carbohydrate (e.g., glucose) and encapsulated or coated vitamin particles in a single container. FIG. 1F shows a media kit comprising agglomerated bulk media, and agglomerated or powdered carbohydrate (e.g., glucose) and vitamins in a single container.

FIG. 2A-B show two media types (Efficient Feed C+[EFC+] and Dynamis, Gibco, Thermo Fisher Scientific) with (cGMP) and without a carbohydrate source (-Glucose) in a sealed container (Securitainer, Thermo Fisher Scientific) or in a vacuum sealed bag at 8 days at 4° C. or 37° C. (FIG. 2A) or 95 days at 4° C. or 37° C. (FIG. 2B). Pictures indicated with an asterisk show significant appearance change consistent with Maillard-reaction induced degradation.

FIG. 3A-F show viability (FIG. 3A, FIG. 3D), viable cell density (VCD; FIG. 3B, FIG. 3E), and IgG titer (FIG. 3C, FIG. 3F) of ExpiCHO S (6C9) cells in Dynamis medium with Efficient Feed C+(EFC+, Gibco, Thermo Fisher Scientific) after storage for 40-days at 4° C. (FIG. 3A-C) or 37° C. (FIG. 3D-F). The media and conditions correspond to those in FIG. 2A-B.

FIG. 4A-D show viability (FIG. 4A, FIG. 4C) and viable cell density (VCD; FIG. 4B, FIG. 4D) of ExpiCHO S (6C9) cells in Dynamis medium with Efficient Feed C+(EFC+, Gibco, Thermo Fisher Scientific) after storage for 6-months at 4° C. (FIG. 4A-B) or 25° C. (FIG. 4C-D).

FIG. 5A-D show IgG production (FIG. 5A, FIG. 5C) and specific production (FIG. 5B, FIG. 5D) of ExpiCHO S (6C9) cells in Dynamis medium with Efficient Feed C+(EFC+, Gibco, Thermo Fisher Scientific) after storage for 6-months at 4° C. (FIG. 5A-B) or 25° C. (FIG. 5C-D).

FIG. 6A-D show viability (FIG. 6A, FIG. 6C) and viable cell density (VCD; FIG. 6B, FIG. 6D) of CHOK1 (HER) cells in Dynamis medium with Efficient Feed C+(EFC+, Gibco, Thermo Fisher Scientific) after storage for 6-months at 4° C. (FIG. 6A-B) or 25° C. (FIG. 6C-D).

FIG. 7A-D show protein product production (FIG. 7A, FIG. 7C) and specific production (FIG. 7B, FIG. 7D) of CHOK1 (HER) cells in Dynamis medium with Efficient Feed C+(EFC+, Gibco, Thermo Fisher Scientific) after storage for 6-months at 4° C. (FIG. 7A-B) or 25° C. (FIG. 7C-D).

FIG. 8 shows HEK cell growth in RT-stable DYNAMIS AGT formulations when stored at 37° C. for 78 days. a. Viability (%) of HEK293 (1F11) cells cultured in GMP or various RT-stable (RAD) DYNAMIS formulations stored at 37° C. for 78 days. Control is GMP DYNAMIS stored at 4° C. for 78 days. All conditions were performed in triplicate. Error bars are SEM. b. Viable cell density (VCD) of HEK293 (1F11) cells cultured in GMP or various RT-stable (RAD) DYNAMIS formulations stored at 37° C. for 78 days. Control is GMP DYNAMIS stored at 4° C. for 78 days. All conditions were performed in triplicate. Error bars are SEM.

FIG. 9 shows CHO cell growth and performance in RT-stable DYNAMIS and EFFICIENTFEED C+(EFC+) AGT formulations stored at room temperature for 13 months. ExpiCHO S (a, b) and CHO K1 (c, d) cell lines were cultured in Standard (GMP) or RT-stable DYNAMIS/EFC+AGT stored at room temperature (20° C.-25° C.) for 13 months. Viable cell density (VCD, a and b) was measured using the ViCell Blu. Titer (c, d) was measured on D14 using the Cedex BioHT. Control was cells cultured in Standard (GMP) DYNAMIS/EFC+ that were stored at 4° C. for 13 months. All conditions were performed in triplicate. Error bars are SEM.

### DETAILED DESCRIPTION

### General Overview

The present disclosure relates to formulations of cell culture media and supplements that are stable when stored at room temperature (e.g., about 15° C.-30° C.). A typical cell culture requires sugar sources such as glucose and vitamins. However, sugar sources and some vitamins must be stored at refrigerated temperatures (e.g., 2° C.-8° C.) to prevent oxidation of the sugars, vitamins, and other media components that the sugars and/or vitamins may be in contact with. The formulations described herein advantageously allow for cell culture media and supplements to be stored at room temperature without causing oxidation or degradation. Various compositions, kits and formulations are described. These various embodiments segregate the carbohydrates and/or vitamins from the other components and prevent interaction or reaction, thus improving the stability of the media or supplement at room temperature. Storing cell culture media and supplements at room temperature allows for freeing up valuable refrigerator space and enabling a substantial decrease in energy usage. Moreover, the formulations described herein are able to be stored anywhere and are easy to use.

### Definitions

Unless otherwise defined, all technical and scientific terms used herein have the same meaning as commonly understood by one of ordinary skill in the art. For example, any nomenclatures used in connection with, and techniques of cell and tissue culture, biochemistry, molecular biology, immunology, microbiology, genetics, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to +10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If one or both end points are modified by the term “about,” the range specified is expanded by a variation of up to +10% of any value within the range or within 3 or more standard deviations, including the end points.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the terms “room temperature,” “RT,” or “ambient temperature” refer the to typical temperature in an indoor laboratory setting. In one aspect, the laboratory setting is climate controlled to maintain the temperature at a substantially uniform temperature or with a specific range of temperatures. In one aspect, “room temperature” refers a temperature of about 15-30° C., including all integers and endpoints within the specified range. In another aspect, “room temperature” refers a temperature of about 15-30° C.; about 20-30° C.; about 22-30° C.; about 25-30° C.; about 27-30° C.; about 15-22° C.; about 15-25° C.; about 15-27° C.; about 20-22° C.; about 20-25° C.; about 20-27° C.; about 22-25° C.; about 22-27° C.; about 25-27° C.; about 15° C.±10%; about 20° C.±10%; about 22° C.±10%; about 25° C.±10%; about 27° C.±10%; ˜ 20° C., ˜22° C., ˜25° C., or ˜27° C., at standard atmospheric pressure.

As used herein, the phrases “room temperature-stable” or “stable at room temperature” refers to the ability of cell culture media to resist degradation, oxidation, or reduction over a period of time at room temperature as described herein.

As used herein the phrase “stable . . . over a period of time” refers to the time period over which a room temperature-stable cell culture media will resist or be prevented from degradation, oxidation, or reduction at room temperature. The period of time can be from 1 day to 2 years, including all integers and dimensions of time (days, weeks, months, or years) within the specified range. The media may be stable for over 30 days, 60 days, 90 days, 120 days, 150 days, 180 days, 210 days, 240 days, 270 days, 300 days, 330 days, 360 days, 390 days, 420 days, 450 days, 480 days, 510 days, 540 days, 570 days, 600 days, 630 days, 660 days, 690 days, 720 days, 730 days or greater than 730 days. The media may be stable for over 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, or greater than 24 months. The media may be stable for over 0.1 of a year, 0.25 of a year, 0.5 of a year, 0.75 of a year, 1 year, 1.1 of a year, 1.25 of a year, 1.5 of a year, 1.75 of a year, 2 years, or greater than 2 years.

The term “extended period of time” is meant a period of time longer than that for which the sample (e.g., nutritive medium, medium supplement, medium subgroup, or buffer) is stored. As used herein, an “extended period of time” therefore means about 1-36 months, about 2-30 months, about 3-24 months, about 6-24 months, about 9-18 months, or about 4-12 months, under a given storage condition, which may include storage at temperatures of about −70° C. to about 37° C., about −20° C. to about 37° C., about 0° C. to about 37° C., about 4° C. to about 37° C., about 4° C. to about 25° C., about 10° C. to about 25° C., or about 20° C. to about 25° C. Assays for determining the biological or biochemical activity of pharmaceutical or clinical compositions, cell culture reagents, nutrients, nutritive media, media supplement, media subgroup, or buffers are well known in the art and are familiar to one of ordinary skill.

The terms “segregate” and “separated,” as used herein refer to limiting the ability of medium components to contact and/or interact with each other. Separation can include physical separation in individual containers or in containers with multiple compartments, such as divided pails, pouches, bags, or the like. For example, in one aspect, a segregated formulation may contain the base medium in one container, the carbohydrates in another container, and the vitamins in a third container. Alternatively, layering the carbohydrates and/or vitamins over or under the base medium in a single container would segregate or separate them by limiting the ability to interact only at the layer interface(s) where the components are in contact with each other.

The term “situated” as used herein refers to placing or storing a component in a receptacle or container.

The term “layered” as used herein refers to placing or storing one or more components over or under other components in a single receptacle or container where the components only contact each other at the interface between the two (or more) layers.

The terms “agglomerated,” “Advanced Granulation Technology,” or “AGT” (Gibco) as used herein refer to granular dry media format produced through an advanced manufacturing process to produce complete media formulations in a variety of serum-free, protein-free, and chemically defined media in a dry format. The media is typically prepared using fluid bed technology that produces agglomerated powders having enhanced characteristics (for example, enhanced solubility) from the starting materials. The process consists of suspending powders in an upwardly moving column of air while simultaneously injecting a controlled and defined amount of liquid into the powder stream to produce a moistened state of the powder; mild heat may be then used to dry the material, producing an agglomerated powder. Preparation of agglomerated media, feeds, nutritive powders, supplements, etc., their properties, and methods to prepare auto pH and auto osmolality of agglomerated media, feeds, nutritive powders, supplements, etc. have been described in U.S. Pat. Nos. 6,383,810 and 6,627,426 and U.S. Pat. App. Pub. No. US 2019/0048312 A1, inter alia, each of which is incorporated by reference for teachings related to agglomerated media.

The term “powder” or “dry powder” as used herein refers to media powders or powdered media compositions for cell culture that are present in dry granular form, whose gross appearance may be free flowing. The term “powder” includes agglomerated powders, as described herein. The term “base powder” or “dry base powder” as used herein refers to a dry powder composition before it is combined with other components.

As used herein, the term “ingredient” refers to any compound, whether of chemical or biological origin, that can be used in cell culture media to maintain or promote the growth of proliferation of cells. The terms “component,” “nutrient” and ingredient” can be used interchangeably and are all meant to refer to such compounds. Typical ingredients that are used in cell culture media include amino acids, salts, metals, sugars, carbohydrates, lipids, nucleic acids, hormones, vitamins, fatty acids, proteins, and the like. Other ingredients that promote or maintain cultivation of cells ex vivo can be selected by those of skill in the art, in accordance with the particular need.

The terms “cell culture” or “culture” as used herein refer to the maintenance of cells in an artificial, e.g., an in vitro environment. It is to be understood, however, that the term “cell culture” is a generic term and may be used to encompass the cultivation not only of individual prokaryotic (e.g., bacterial) or eukaryotic (e.g., animal, plant and fungal) cells, but also of tissues, organs, organ systems or whole organisms, for which the terms “tissue culture,” “organ culture,” “organ system culture” or “organotypic culture” may be used interchangeably with the term “cell culture.”

The phrases “cell culture medium,” “culture medium,” “medium formulation,” or “medium” (plural “media” in each case) as used herein refer to a nutritive solution that supports the cultivation and/or growth of cells; these phrases may be used interchangeably. A cell culture medium may be a basal medium (a general medium that requires additional ingredients to support cell growth) or a complete medium that has all or almost all components to support cell growth. Cell culture media may be serum-free, protein-free (one or both), may or may not require additional components like growth factors, additives, feeds, supplements, for efficient and robust cell performance.

Nutritive media can also be divided into various “subgroups” that can be prepared and used as described herein. Such subgroups can be combined to produce a nutritive medium. For examples of compatible subgroups and related considerations, see U.S. Pat. Nos. 5,474,931 and 5,681,748, which are incorporated by reference herein for such teachings.

The term “combining” as used herein refers to the mixing or admixing of ingredients in a cell culture medium formulation. Combining can occur in liquid or powder form or with one or more powders and one or more liquids. In another example, two or more powdered components may be mixed and then agglomerated to produce a complex mixture such as media, media supplements, media subgroups, or buffers. Combining also includes mixing dry components with liquid components.

The phrases “concentrate” or “concentrated” are used interchangeably and refer to a component or group of components that are at a concentration higher than that desired in the cell culture medium to be supplemented with the concentrate.

The term “contacting” as used herein refers to the placing of cells to be cultivated into a culture vessel with the medium in which the cells are to be cultivated. The term “contacting” encompasses inter alia mixing cells with medium, perfusing cells with medium, pipetting medium onto cells in a culture vessel, and submerging cells in culture medium. “Contacting” or “in contact with each other” as used herein with regard to specific medium formulations refers to the individual components such as base medium, carbohydrates, and vitamins that are in physical contact with one another and permits the components to interact or react with each other. An admix of components would be in contact with one another.

The term “cultivation” as used herein refers the maintenance of cells in an artificial environment under conditions favoring growth, differentiation, or continued viability, in an active or quiescent state, of the cells. Thus, “cultivation” may be used interchangeably with “cell culture” or any of its synonyms described above.

The term “culture vessel” as used herein refers to a receptacle for holding cells. The vessel may be glass, plastic, metal, or other material that can provide an aseptic environment for culturing, cultivating, holding, or storing cells.

The term “extract” as used herein refers to a composition comprising or concentrated preparation of the subgroups of a substance, typically formed by treatment of the substance either mechanically (e.g., by pressure treatment) or chemically (e.g., by distillation, precipitation, enzymatic action, or high salt treatment).

The term “effective amount” or “effective concentration” refers to an amount of an ingredient, which is available for use. One example is the amount of a vitamin in a culture medium, which is available to cells for use in biological processes normally associated with that vitamin. Thus, an effective amount includes the amount of a cell culture ingredient (e.g., a vitamin or sugar) available for a cell to metabolize. An effective amount of an ingredient can be determined, for example, from the knowledge available to one skilled in the art and/or by experimental determination.

A “feed” or “supplement” as used herein refers to a composition when added to cells in standard culture may be beneficial for its maintenance, or expansion, or growth, or viability, or affects its cell performance, or increases culture longevity or maintaining cells in a pseudo-stationary phase wherein product expression continues, or results in a significant increase in final product titer. A feed or supplement may be used interchangeably in this disclosure and refers to and liquid formats (including agglomerated formats) of media components comprising one or more amino acids, sugars, vitamins, buffers, sometimes, peptides, hydrolysates, fractions, growth factors, hormones, etc. required to rebalance or replenish or to modulate the growth or performance of a cell in culture, or a cell culture system. A feed or supplement may be distinguished from a cell culture medium in that it is added to a cell culture medium that can culture a cell. As would be understood by one of skill in the art, sometimes a feed/supplement may comprise those amino acids, sugars, vitamins, buffers, etc. required to rebalance or replenish or modulate the growth or performance of a cell in culture, or a cell culture system. A feed or supplement may or may not be concentrated or may be partially concentrated for certain components only.

A cell culture medium is composed of many ingredients and these ingredients vary from one culture medium to another. A “1× formulation” is meant to refer to any aqueous solution that contains some or all ingredients found in a cell culture medium at working concentrations. The “1× formulation” can refer to, for example, the cell culture medium or to any subgroup of ingredients for that medium. The concentration of an ingredient in a 1× solution is about the same as the concentration of that ingredient found in a cell culture formulation used for maintaining or cultivating cells in vitro. A cell culture medium used for the in vitro cultivation of cells is a 1× formulation by definition. When a number of ingredients are present, each ingredient in a 1× formulation has a concentration about equal to the concentration of those ingredients in a cell culture medium. For example, RPMI-1640 culture medium contains, among other ingredients, 0.2 g/L l-arginine, 0.05 g/L I-asparagine, and 0.02 g/L I-aspartic acid. A “1× formulation” of these amino acids contains about the same concentrations of these ingredients in solution. Thus, when referring to a “1× formulation,” it is intended that each ingredient in solution has the same or about the same concentration as that found in the cell culture medium being described. The concentrations of ingredients in a 1× formulation of cell culture medium are well known to those of ordinary skill in the art. See Banes et al., Methods for Preparation of Media, Supplements and Substrate for Serum-Free Animal Cell Culture, Alan R. Liss, N.Y. (1984), which is incorporated by reference herein in its entirety. The osmolality and/or pH, however, may differ in a 1× formulation compared to the culture medium, particularly when fewer ingredients are contained in the 1× formulation. The 1× concentration of any component is not necessarily constant across various media formulations. 1× might therefore indicate different concentrations of a single component when referring to different media. However, when used, 1× will indicate a typical working concentration commonly found in the types of media being referenced. A 1× amount is the amount of an ingredient that will result in a 1× concentration for the relevant volume of medium.

A “10× formulation” as used herein refers to a solution wherein each ingredient in that solution is about 10-times more concentrated than the same ingredient in the cell culture medium. For example, a 10× formulation of RPMI-1640 culture medium may contain, among other ingredients, 2.0 g/L l-arginine, 0.5 g/L I-asparagine, and 0.2 g/L I-aspartic acid (cf. the 1× formulation, above). A “10× formulation” may contain a number of additional ingredients at a concentration about 10 times that found in the 1× culture medium. As will be readily apparent, “5× formulation,” “20× formulation,” “25× formulation,” “50× formulation” and “100× formulation” designate solutions that contain ingredients at about 5-, 20-, 25-, 50- or 100-fold concentrations, respectively, as compared to a working 1× cell culture medium. Again, the osmolality and pH of the media formulation and concentrated solution may vary. See U.S. Pat. No. 5,474,931, which is directed to culture media concentrate technology and is incorporated by reference herein for such teachings.

As used herein “physiologic pH” is greater than about 4 and less than about 9. Other or particular pH values or ranges, e.g., minimum or maximum pHs of greater than 4.2, 4.5, 4.8, 5.0, 5.2, 5.5, 5.7, 5.8, 6.0, 6.2, 6.5, 6.7, 6.8, 7.0, 7.2, 7.4, 7.5, 7.8, 8.0, 8.2, 8.4, 8.5, 8.7, 8.8, etc. or from about 4.0 to about 9.0, from about 4.0 to about 5.0, from about 5.0 to about 6.0, from about 6.0 to about 7.0, from about 8.0 to about 9.0, from about 4.0 to about 6.0, from about 5.0 to about 7.0, from about 6.0 to about 8.0, from about 7.0 to about 9.0, from about 6.0 to about 9.0, or from about 4.0 to about 7.0 may also be used for dissolving supplements. Some supplements, though not preferred, may only be entirely soluble outside these ranges. In some aspect, “physiologic pH” is about pH 7.4±10%.

An “auto-pH” or “auto-pHing” medium, medium supplement, or buffer as described herein is a formulation which has been formulated such that, upon rehydration with a solvent, the resulting medium, medium supplement, or buffer solution is at a desired pH and does not require adjustment of the pH with acid or base prior to use. For example, an auto-pH culture medium that is formulated to be used at pH 7.4 will, upon rehydration with a solvent, be at pH 7.4 and therefore will be ready for immediate use without further adjustment of the pH.

The phrase “without significant loss of biological and biochemical activity” as used herein refers to a decrease of less than about 30%, preferably less than about 25%, more preferably less than about 20%, still more preferably less than about 15%, and most preferably less than about 10%, of the biological or biochemical activity of the nutritive media, media supplement, media subgroup, buffer, or sample of interest when compared to a freshly made nutritive media, media supplement, media subgroup, buffer, or sample of the same formulation.

As used herein a “solvent” is a liquid that dissolves or has dissolved another ingredient of the medium. Solvents may be used in preparing media, in preparing subgroups, supplements or other formulations, and in reconstituting media or diluting a concentrate in preparation for culturing cells. Solvents may be polar, e.g., an aqueous solvent, or non-polar, e.g., an organic solvent. Solvents may be complex, i.e., requiring more than one ingredient to solubilize an ingredient. Complex solvents may be simple mixtures of two liquids such as alcohol and water or may be mixtures of salts or other solids in a liquid. Two, three, four, five, six, or more components may be necessary in some cases to form a soluble mixture. Simple solvents such as mixtures of ethanol or methanol and water are preferred because of their ease of preparation and handling.

### Physical Separation of Media Components

In the present disclosure, the inventors investigated whether removing glucose from cell culture media would help to reduce the degradation that is seen when storing the cell culture media at room-temperature. Herein, the inventors were able to show that when media is stored separately from its carbohydrate source (e.g., glucose) at room-temperature that it exhibits significantly less degradation compared to complete media.

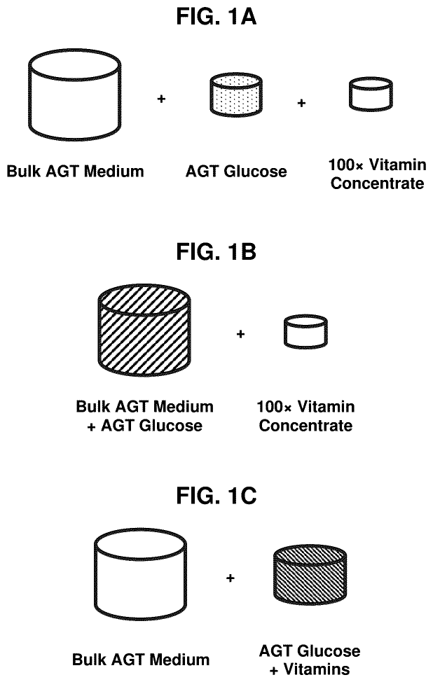

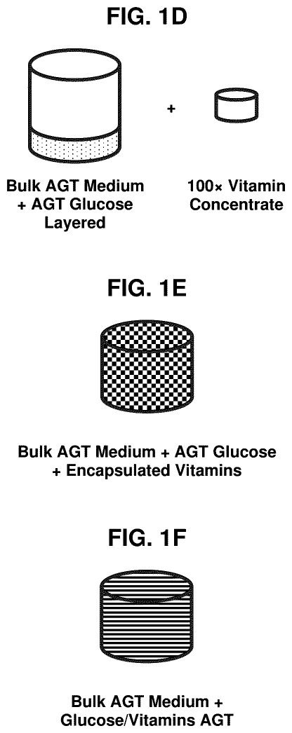

In one embodiment, the media components are physically separated as shown in FIGS. 1A and 1C. In FIG. 1A, the media (e.g., basic media that includes amino acids and trace elements), a carbohydrate source (e.g., glucose) and vitamins are provided in separate containers or pouches wherein the vitamin concentrate is refrigerated (about 2-8° C.). The example shown in FIG. 1A illustrates this by showing bulk AGT medium, AGT glucose, and a vitamin concentrate.

In another embodiment the media components are physically divided into media (e.g., basic media that includes amino acids and trace elements) and a separate carbohydrate source (e.g., glucose) with vitamins. This is illustrated in FIG. 1C which shows bulk AGT medium in one container and AGT glucose and vitamins in another container. In one embodiment, the vitamins are mixed together with sugars (e.g., glucose).

Alternatively, any other basic and glucose-free media that contains amino acids and trace elements could be used here and the carbohydrate source could also be powdered. In yet another alternative embodiment, the vitamins could be encapsulated.

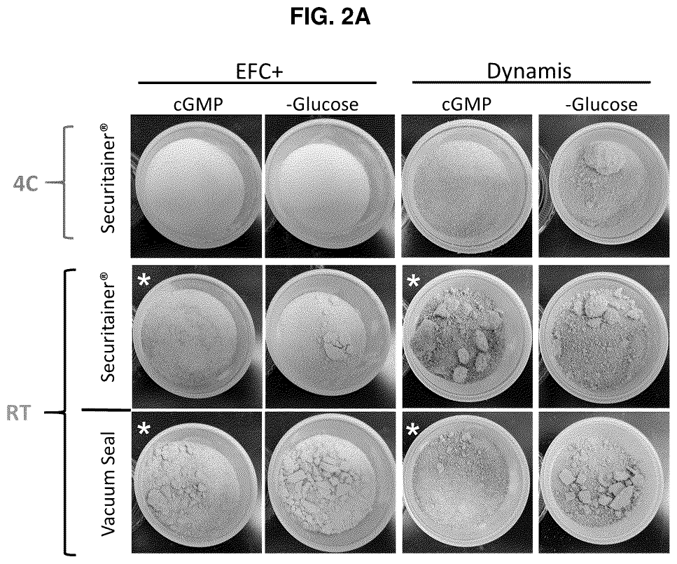

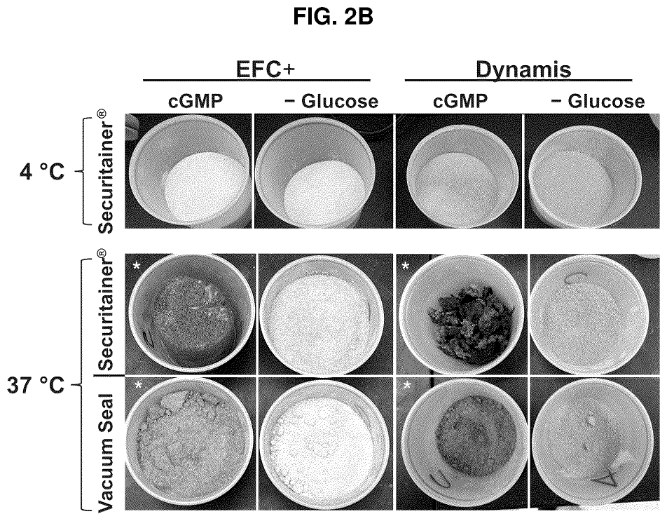

Typical Maillard reactions occur quickly and at high temperatures (i.e. a 350° F. oven), but it has been shown this reaction still occurs at lower temps (such as room temperature) over a longer period. While there are likely a multitude of molecular reactions occurring between the components of complex cell culture media, it is hypothesized that the Maillard reaction could be, in-part, responsible for some of the degradation seen when cell culture media is stored at room-temperature. Thus, the inventors investigated whether removing glucose (and thus, inhibiting the Maillard reaction) would help to remedy any of the degradation of room-temperature stored media. The inventors showed that when media is stored separately from its carbohydrate source at room temperature that it exhibits significantly less degradation of many of its components compared to complete media which contains sugars such as glucose. Since cell culture media stored at ambient temperature is not heated, it was surprising that the removal of glucose resulted in considerably less degradation (see FIG. 2B) suggesting that the Maillard reaction is likely a major contributor to degradation in cell culture media (containing sugar) stored at ambient temperatures.

Interestingly, although removal of glucose resulted in less degradation of the majority of cell culture media components, there was considerably more degradation seen in certain vitamins that remained in the media. The inventors have further surprisingly shown that when vitamins are also removed from the media and separately stored with the carbohydrate source (e.g., mixed together with glucose) there is less degradation than when vitamins are part of a complete medium. This was not expected since it was believed that most vitamins (which contain amine groups) would be degraded in the presence of glucose. At ambient temperatures, carbohydrate sources such as sugars can engage in Maillard reactions with other components, such as amino acids and vitamins, and degrade these components. Without wishing to be bound by theory, the inventors speculate that the reduced degradation is the result of less exposure between the vitamins and other components of cell culture media (amino acids, trace elements, etc.) that react adversely with one another. This is also true under ambient temperatures when the Maillard reaction creates MRPs that react adversely with media components including vitamins. Vitamins that are separately stored with a carbohydrate source include, but are not limited to, vitamins shown in Table 2.

### Compositions

Some embodiments described herein are compositions for nutritive media, cell culture media, feeds, media supplements, media subgroups, or buffer; methods for producing nutritive media, media supplements, feeds, media subgroups, or buffers; and the media products produced thereby. Nutritive media, media supplements and media subgroups produced as described herein include any media, media supplement or media subgroup (serum-free or serum-containing) which may be used to support the growth of a cell, which may be a bacterial cell, a fungal cell (particularly a yeast cell), a plant cell or an animal cell (particularly an insect cell, a nematode cell or a mammalian cell, most preferably a human cell), any of which may be a somatic cell, a germ cell, a normal cell, a diseased cell, a transformed cell, a mutant cell, a stem cell, a precursor cell or an embryonic cell. Preferred nutritive media include, but are not limited to, cell culture media, most preferably a bacterial cell culture medium, plant cell culture medium, or animal cell culture medium. Preferred media supplements include, but are not limited to, undefined supplements such as extracts of bacterial, animal or plant cells, glands, tissues or organs, particularly bovine pituitary extract, bovine brain extract and chick embryo extract; and biological fluids (particularly animal sera, and most preferably bovine serum, particularly fetal bovine, newborn calf or normal calf serum, horse serum, porcine serum, rat serum, murine serum, rabbit serum, monkey serum, ape serum or human serum, any of which may be fetal serum) and extracts thereof (more preferably serum albumin and most preferably bovine serum albumin or human serum albumin). Medium supplements may also include defined replacements such as StemPro LipoMAX, OptiMAb, Knock-Out Serum Replacement (GIBCO, Thermo Fisher Scientific Inc.), and the like, which can be used as substitutes for the undefined media supplements described above. Such supplements may also comprise defined components, including but not limited to, hormones, cytokines, neurotransmitters, lipids, attachment factors, proteins, and the like.

In one embodiment, the media, media supplements, media subgroups, or buffers, comprises agglomerated powders of media, media supplements, media subgroups, or buffers. In one aspect described herein, the agglomerated nutritive media, media supplements, media subgroups, and buffers are produced using fluid bed technology to agglomerate the solutions of media, media supplements, media subgroups, or buffers. Fluid bed technology is a process of producing agglomerated powders having altered characteristics (particularly, for example, solubility) from the starting materials. In general applications of the technology, powders are suspended in an upwardly moving column of air while at the same time a controlled and defined amount of liquid is injected into the powder stream to produce a moistened state of the powder; mild heat is then used to dry the material, producing an agglomerated powder. In one aspect, the agglomerated media, media supplements, media subgroups, or buffers is produced using the proprietary Advanced Granulation Technology (AGT dry media format) (GIBCO). See Jayme et al., “A Novel Application of Granulation Technology to Improve Physical Properties and Biological Performance of Powdered Serum-Free Culture Media,” In: Shirahata et al. (eds) Animal Cell Technology: Basic & Applied Aspects, Vol. 12 (2002), Springer, Dordrecht. Specific commercially available agglomerated media, supplements, feeds, and additives include CD CHO AGT, CD OptiCHO AGT, CD FortiCHO AGT, DYNAMIS AGT, VP-SFM AGT, OptiPro AGT, CD Hybridoma AGT, CHO CD EFFICIENTFEED A, B and C AGT, CHO CD EFFICIENTFEED A+, B+ and C+AGT Nutrient Supplement, and FunctionMAX TiterEnhancer Additive (all from GIBCO; the respective product descriptions are each incorporated by reference herein for such teachings).

The formulations and methods described herein can be used to prepare any nutritive media, media supplement, media subgroup, or buffer and stored for an extended period of time without significant loss of biological or biochemical activity.

Any nutritive medium, medium supplement, medium subgroup, or buffer may be prepared by the methods described herein. Particularly preferred nutritive media, media supplements, and media subgroups that may be prepared as described herein include cell culture media, media supplements, and media subgroups that support the growth of animal cells, plant cells, bacterial cells, or yeast cells. Particularly preferred buffers that may be prepared as described herein include balanced salt solutions, which are isotonic for animal cells, plant cells, bacterial cells, or yeast cells.

Examples of animal cell culture media that may be prepared as described herein include, but are not limited to, DMEM, RPMI-1640, MCDB 131, MCDB 153, MDEM, IMDM, MEM, M199, McCoy's 5A, Williams' Media E, Leibovitz's L-15 Medium, Grace's Insect Medium, IPL-41 Insect Medium, TC-100 Insect Medium, Schneider's Drosophila Medium, Wolf & Quimby's Amphibian Culture Medium, F10 Nutrient Mixture, F12 Nutrient Mixture, and cell-specific serum-free media (SFM) such as those designed to support the culture of keratinocytes, endothelial cells, hepatocytes, melanocytes, CHO cells, 293 cells, PerC6, hybridomas, hematopoetic cells, embryonic cells, neural cells etc. Specific chemically defined media products include CD CHO Medium (GIBCO), CD OptiCHO Medium (GIBCO), EX-CELL Advanced CHO Medium (Millipore Sigma-Aldrich), HyClone ActiPro (GE Healthcare Life Sciences). Specific feed supplements include CHO CD EfficientFeed A (or B) AGT Nutritional Supplement (GIBCO), CD EfficientFeed C AGT Nutrient Supplement (GIBCO), EfficientFeed A+AGT Supplement (GIBCO), EfficientFeed B+AGT Supplement (GIBCO), Resurge CD1 Supplement (GIBCO), HyClone Cell Boost Supplements (various versions) (GE Healthcare Life Sciences), EX-CELL Advanced CHO Feed 1 (Millipore Sigma-Aldrich), et al. Other media, media supplements, and media subgroups suitable for preparation are available commercially. Formulations for these media, media supplements and media subgroups, as well as many other commonly used animal cell culture media, media supplements and media subgroups are known in the art and available from commercial suppliers, e.g., Thermo Fisher Scientific Inc., Life Technologies, GIBCO, INVITROGEN, et al.

Examples of plant cell culture media that may be prepared as described herein include, but are not limited to, Anderson's Plant Culture Media, CLC Basal Media, Gamborg's Media, Guillard's Marine Plant Culture Media, Provasoli's Marine Media, Kao and Michayluk's Media, Murashige and Skoog Media, Mccown's Woody Plant Media, Knudson Orchid Media, Lindemann Orchid Media, or Vacin and Went Media. Formulations for these media, which are commercially available, as well as for many other commonly used plant cell culture media, are known in the art and available from commercial manufacturers.

Examples of bacterial cell culture media that may be prepared as described herein include, but are not limited to, Trypticase Soy Media, Brain Heart Infusion Media, Yeast Extract Media, Peptone-Yeast Extract Media, Beef Infusion Media, Thioglycollate Media, Indole-Nitrate Media, MR-VP Media, Simmons' Citrate Media, CTA Media, Bile Esculin Media, Bordet-Gengou Media, Charcoal Yeast Extract (CYE) Media, Mannitol-salt Media, MacConkey's Media, Eosin-methylene blue (EMB) media, Thayer-Martin Media, Salmonella-Shigella Media, and Urease Media. Formulations for these media, which are commercially available, as well as for many other commonly used bacterial cell culture media, are well-known in the art and may be found for example in the DIFCO & BBL Manual, 2nd ed. (Becton, Dickinson and Company, 2009) and in the Manual of Clinical Microbiology (American Society for Microbiology, Washington, D.C.).

Examples of fungal cell culture media, particularly yeast cell culture media, which may be prepared as described herein include, but are not limited to, Sabouraud Media and Yeast Morphology Media (YMA). Formulations for these media are commercially available and are known in the art.

One embodiment described herein is a temperature-stable cell culture media composition, media supplement, media subgroup, or kit thereof. Exemplary formulations and kits are shown in Table 1. A minimum composition comprises a base cell culture medium comprising one or more amino acids, salts, buffers, trace minerals, lipids, nucleic acids, or proteins; one or more carbohydrates; and one or more vitamins; wherein the carbohydrates and/or vitamins are segregated from the base cell culture medium. Such segregation can be physical segregation in separate containers or by reducing the ability of the carbohydrates and/or vitamins from interacting or chemically reacting with the base cell culture media in an admixture. Segregation can include situating the various components in separate containers. Segregation can further include, but is not limited to, powdering, granulation, or agglomeration of the carbohydrate and/or vitamins, encapsulating the carbohydrate and/or vitamins, or combinations thereof.

TABLE 1
Exemplary Room-Temperature-Stable Media/Supplement
Formulations and Kits

| | Segregation |
|---|---|
| Components | Compartments* |
| Granulated or agglomerated base media; granulated, | 3 |
| agglomerated, or powdered carbohydrate; 100× | |
| vitamin concentrate | |
| Granulated or agglomerated base media combined with | 2 |
| granulated, agglomerated, or powdered carbohydrate; | |
| 100× vitamin concentrate | |
| Granulated or agglomerated base media; granulated, | 2 |
| agglomerated, or powdered carbohydrate and vitamins | |
| Granulated or agglomerated base media layered over | 2 |
| or under granulated, agglomerated, or powdered | |
| carbohydrate; 100× vitamin concentrate | |
| Granulated or agglomerated base media combined with | 1 |
| granulated, agglomerated, or powdered carbohydrate | |
| and encapsulated or coated vitamins | |
| Granulated or agglomerated base media combined with | 1 |
| granulated, agglomerated, or powdered carbohydrate | |
| and vitamins | |

*The number of segregation compartments does not necessarily mean separate containers. For example, a divided pail, pouch, or bag could segregate two or three components, but be a single container.

An exemplary media formulation is provided in Table 2. The exemplary media composition can be formulated as a liquid or dry powder that is reconstituted or dissolved in water prior to use. Liquid components, such as fetal bovine serum, are typically added after dissolution of the dry powder. The media can also be prepared as agglomerated granules using fluid bed technology. Dry powder media and agglomerated media can be prepared as described herein.

TABLE 2
Exemplary Media or Supplement Formulations

| | | Exemplary Mass |
|---|---|---|
| Component | Exemplary Species | Percent (%) |
| Carbohydrate(s) | glucose, fructose, trehalose, etc. | 20-65 |
| (carbon source) | | |
| Vitamins | retinol (A), thiamine (B1), riboflavin | 1-5 |
| | (B2), niacinamide (B3), pantothenic | |
| | acid (B5), pyridoxamine (B6), biotin | |
| | (B7), folic acid (B9) cobalamin (B12), | |
| | ascorbic acid (C), cholecalciferol (D), | |
| | tocopherol (E), phylloquinone (K), | |
| | choline, inositol, lipoic acid, | |
| | paraaminobenzoic acid | |

Base Media

| | | |
|---|---|---|
| Amino Acids | alanine, arginine, aspartic acid, | 20-40 |
| | glutamic acid, glycine, histidine, | |
| | isoleucine, methionine, phenylalanine, | |
| | proline, hydroxyproline, serine, | |
| | threonine, tryptophan, valine, | |
| | tyrosine, cysteine, lysine | |
| Inorganic salts, | salts of sodium, potassium, | 2-10 |
| buffers | magnesium, calcium, ammonium, | |
| | phosphate, carbonate, sulfate | |
| Trace minerals | iron, manganese, copper, iodine, zinc, | 0.01-0.05 |
| | cobalt, fluoride, chromium, | |
| | molybdenum, selenium, nickel, | |
| | silicon, vanadium | |
| Additives | antibiotics, pH indicators, surfactants, | 0.1-5 |
| | amines, lipids, etc. | |
| Proteins: Serum or | fetal bovine serum (FBS), serum | 1-10 |
| serum replacement | replacement (e.g., KnockOut, Gibco) | |

The exemplary media or supplement formulation shown in Table 2 can be formulated in various formulations and kits as shown in Table 1.

Any of the above media, media supplement, media subgroup, or buffer that can be prepared as described herein may also include one or more additional components, such as indicating or selection agents (e.g., dyes, antibiotics, amino acids, enzymes, substrates, and the like), filters (e.g., charcoal), salts, polysaccharides, ions, detergents, stabilizers, and the like. The embodiment is not limited to presently formulated media but is broadly applicable to any media formulation or supplement for culturing cells.

In another embodiment described herein, the culture media may comprise one or more buffer salts, preferably sodium bicarbonate, at concentrations sufficient to provide optimal buffering capacity for the culture medium. In one aspect the one or more buffer comprises acetic acid, acetylsalicylic acid, adipic acid, alginic acid, ascorbic acid, aspartic acid, benzoic acid, benzenesulfonic acid, bisulfic acid, boric acid, butanoic acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, gly-glycine, gluco heptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthalenesulfonic acid, naphthilic acid, nicotinic acid, nitrous acid, oxalic acid, pelargonic, phosphoric acid, propionic acid, pyruvic acid, saccharin, salicylic acid, sorbic acid, succinic acid, sulfuric acid, tartaric acid, thiocyanic acid, thioglycolic acid, thiosulfuric acid, tosylic acid, undecylenic acid, MES, bis-tris methane, ADA, ACES, bis-tris propane, PIPES, MOPSO, cholamine chloride, MOPS, BES, TES, HEPES, DIPSO, MOBS, acetamido glycine, TAPSO, TEA, POPSO, HEPPSO, EPS, HEPPS, Tricine, Tris(hydroxymethyl)aminomethane (tromethamine), glycinamide, glycylglycine, HEPBS, Bicine, TAPS, AMPB, CHES, AMP, AMPSO, CAPSO, CAPS, CABS, combinations thereof, or salts thereof. In one aspect, the buffer comprises one or more of phosphate, sulfate, carbonate, formate, acetate, propionate, butanoate, lactate, glycine, maleate, pyruvate, citrate, aconitate, isocitrate, α-ketoglutarate, succinate, fumarate, malate, oxaloacetate, aspartate, glutamate, tris(hydroxymethyl)aminomethane (tromethamine), combinations thereof, or salts thereof. In one aspect, the buffer is sodium carbonate or sodium bicarbonate.

According to one aspect described herein, a buffer salt, such as sodium bicarbonate, may be added to the medium prior to, during, or following agglomeration of the medium. In one example of this aspect described herein, the sodium bicarbonate may be added to the culture medium prior to, during or following agglomeration with an appropriate solvent (such as water, serum or a pH-adjusting agent such as an acid (e.g., HCl at a concentration of 1 M to 5 M, 0.1 M to 5 M, or preferably at 1 M) or a base (e.g., NaOH at a concentration of 1 M to 5 M, 0.1 M to 5 M, or preferably at 1 M) such that, upon reconstitution of the agglomerated medium the culture medium is at the optimal or substantially optimal pH for cultivation of a variety of cell types. For example, bacterial cell culture media prepared by the present methods will, upon reconstitution, preferably have a pH of about 4-10, more preferably about 5-9 or about 6-8.5. Fungal (e.g., yeast) cell culture media prepared by the present methods will, upon reconstitution, preferably have a pH of about 3-8, more preferably about 4-8 or about 4-7.5; animal cell culture media prepared by the present methods will, upon reconstitution, preferably have a pH of about 6-8 or about 7-8, more preferably about 7-7.5, or about 7.2-7.4; and plant cell culture media prepared by the present methods will, upon reconstitution, preferably have a pH of about 4-8, preferably about 4.5-7, 5-6 or 5.5-6. Optimal pH for a given culture medium to be used on a particular cell type may also be determined empirically by one of ordinary skill using art-known methods. For example, gastric cells may be cultured at pHs well below those of other cells, for example, pH 1-3. One of ordinary skill appreciates that other cells adapted to harsh environments may have special tolerances or needs that might be outside the normal ranges that satisfy culture conditions for commonly cultured cells.

In another example, one or more buffer salts, e.g., sodium bicarbonate, may be added directly to a nutritive medium. In a related aspect, a pH-adjusting agent such as an acid (e.g., HCl) or a base (e.g., NaOH) may be added to a nutritive medium, which may contain one or more buffer salts (such as sodium bicarbonate), by agglomeration of the pH-adjusting agent into nutritive medium in a fluid bed apparatus, by spray-drying the pH-adjusting agent onto the powdered or agglomerated nutritive medium, or by a combination thereof; this approach obviates the subsequent addition of a pH-adjusting agent after reconstitution of the powdered medium. Thus, the nutritive culture medium described herein is useful in cultivation or growth of cells in vitro that, upon reconstitution with a solvent (e.g., water or serum), has a pH that is optimal for the support of cell cultivation or growth without a need for adjustment of the pH of the liquid medium. This type of medium, defined herein as “automatically pH-adjusting medium,” therefore obviates the time-consuming and error-prone steps of adding buffer(s) to the medium after reconstitution and adjusting the pH of the medium after dissolution of the buffer(s). For example, a mammalian cell culture medium prepared according to these methods may, upon reconstitution, have a pH of between about 7.1 to about 7.5, more preferably between about 7.1 to about 7.4, and most preferably about 7.2 to about 7.4 or about 7.2 to about 7.5.

In another embodiment, automatically pH-adjusting media can be produced by preparing reconstituted media without the addition of any buffering systems or pH-adjusting agents. In a preferred such aspect, an auto-pH medium may be provided by adjusting the buffering systems present in the medium. For example, as one of ordinary skill is aware, culture media typically contain buffers or buffering systems. By adjusting the pH-opposing forms of such buffers in the medium, an auto-pH medium is created, avoiding the requirement to add additional buffers or pH-adjusting agents to achieve a proper pH level prior to or upon reconstitution of the medium and prior to use. In one such aspect described herein, pH-opposing forms of certain media components (particularly phosphate or other buffer salts) are then used in the culture medium to provide a desired pH upon reconstitution of the powdered media. pH-opposing forms of components are conjugate acid-base pairs in which the members of the pair can either raise the pH or lower it to achieve the desired pH of the solution. Sodium HEPES (pH raising) and HEPES-HCl (pH lowering) are examples of pH opposing components. For example, if a reconstituted media having a pH of between 4.5 and 7.2 is to be prepared, the first step is to determine the correct balance of monobasic (to lower the pH) to dibasic (to raise the pH) phosphate in order to yield the desired pH. Typically, mono- and di-basic phosphate salts are used at concentrations of about 0.1 mM to about 10 mM, about 0.2 mM to about 9 mM, about 0.3 mM to about 8.5 mM, about 0.4 mM to about 8 mM, about 0.5 mM to about 7.5 mM, about 0.6 mM to about 7 mM, or preferably about 0.7 mM to about 7 mM. If other buffer systems are used in the formulations, the proper ratio or balance of the basic (typically sodium or monobasic) buffer salt and the corresponding acidic (or pH-opposing; typically, HCl or dibasic) buffer salt is similarly determined to ensure that the formulation will be at the desired final pH upon reconstitution with a solvent. Because the actual phosphate molecular species that is present in a solution is the same at a given pH whether the basic (e.g., sodium or monobasic) or acidic (e.g., HCl or dibasic) form is added, this adjustment would not be expected to impact buffering capacity. Once an appropriate ratio of pH-opposing forms of an appropriate buffer is determined, these components may be added to the medium (for example, a dry powder medium) to provide a culture medium that is of the appropriate pH level upon reconstitution and prior to use.

In one embodiment, the media, media supplement, media subgroup, or buffer dissolves within about 10-30 minutes in water at 25° C. In one aspect, the time until complete dissolution of a medium (e.g., full dissolution rate or t100) is within about 1 min, about 2.5 min, about 5 min, about 10 min, about 15 min, about 20 min, about 25 min, or about 30 min. In another aspect, the full dissolution rate is within about 2 min, about 3 min, about 4 min, about 5 min, about 6 min, about 7 min, about 8 min, about 9 min, about 10 min, about 11 min, about 12 min, about 13 min, about 14 min, about 15 min, about 16 min, about 17 min, about 18 min, about 19 min, about 20 min, about 21 min, about 22 min, about 23 min, about 24 min, about 25 min, about 26 min, about 27 min, about 28 min, about 29 min, about 30 min, about 31 min, about 32 min, about 33 min, about 34 min, about 35 min, about 36 min, about 37 min, about 38 min, about 39 min, or about 40 min.

In another embodiment, about 50% of the media, media supplement, media subgroup, or buffer dissolves within about 10-30 minutes in water at 25° C. In one aspect, the time until 50% dissolution of a medium (e.g., t50) is within about 1 min, about 2.5 min, about 5 min, about 10 min, about 15 min, about 20 min, about 25 min, or about 30 min. In another aspect, 50% dissolution rate is within about 2 min, about 3 min, about 4 min, about 5 min, about 6 min, about 7 min, about 8 min, about 9 min, about 10 min, about 11 min, about 12 min, about 13 min, about 14 min, about 15 min, about 16 min, about 17 min, about 18 min, about 19 min, about 20 min, about 21 min, about 22 min, about 23 min, about 24 min, about 25 min, about 26 min, about 27 min, about 28 min, about 29 min, about 30 min, about 31 min, about 32 min, about 33 min, about 34 min, about 35 min, about 36 min, about 37 min, about 38 min, about 39 min, or about 40 min. Another embodiment is complete dry powder culture media formulations that support the cultivation of cells in vitro upon reconstitution of the media with a solvent, without the need for the addition of any supplemental nutrient components to the medium prior to use. Media according to this aspect described herein thus will preferably comprise the nutritional components necessary for cultivation of a cell in vitro, such that no additional nutritional components need be included in the solvent or added to the medium upon reconstitution and prior to use. Accordingly, such complete media described herein will be suitable for use in cultivating cells in vitro upon reconstitution with water or with an alternative non-nutrient-containing solvent such as a buffered saline solution. Such complete media may be automatically pH-adjusting media, and may comprise one or more culture medium supplements (including but not limited to serum), one or more amino acids (including but not limited to l-glutamine), insulin, transferrin, one or more hormones, one or more lipids, one or more growth factors, one or more cytokines, one or more neurotransmitters, one or more extracts of animal tissues, organs or glands, one or more enzymes, one or more proteins, one or more trace elements, one or more extracellular matrix components, one or more antibiotics, one or more viral inhibitors, one or more buffers, or combinations thereof.

Examples of media supplements that may be prepared by the present methods, or that may be included in the culture media described herein, include, without limitation, animal sera, such as bovine sera, fetal bovine, newborn calf and calf sera, human sera, equine sera, porcine sera, monkey sera, ape sera, rat sera, murine sera, rabbit sera, ovine sera and the like, defined replacements such as StemPro LipoMAX, OptiMAb, Knock-Out Serum Replacement (Gibco, Thermo Fisher Scientific Inc.), hormones (including steroid hormones such as corticosteroids, estrogens, androgens (e.g., testosterone) and peptide hormones such as insulin, cytokines (including growth factors (e.g., EGF, αFGF, βFGF, HGF, IGF-1, IGF-2, NGF and the like), interleukins, colony-stimulating factors, interferons and the like), neurotransmitters, lipids (including phospholipids, sphingolipids, fatty acids, Excyte, cholesterol and the like), attachment factors (including extracellular matrix components such as fibronectin, vitronectin, laminins, collagens, proteoglycans, glycosaminoglycans and the like), and extracts or hydrolysates of animal, tissues (e.g., plant or bacteria tissues), cells, organs or glands (such as bovine pituitary extract, bovine brain extract, chick embryo extract, bovine embryo extract, chicken meat extract, chicken tissue extract, Achilles tendon and extracts thereof) and the like. Other media supplements that may be produced by the present methods or that may be included in the culture media described herein include a variety of proteins (such as serum albumins, particularly bovine or human serum albumins; immunoglobulins and fragments or complexes thereof; aprotinin; hemoglobin; haemin or haematin; enzymes (such as trypsin, collagenases, pancreatinin, or dispase); lipoproteins; fetuin; ferritin; etc.), which may be natural or recombinant; vitamins; amino acids and variants thereof (including, but not limited to, I-glutamine and l-cysteine), enzyme co-factors; polysaccharides; salts or ions (including trace elements such as salts or ions of molybdenum, vanadium, cobalt, manganese, selenium, and the like); and other supplements and compositions that are useful in cultivating cells in vitro that will be familiar to one of ordinary skill. Media supplements produced by the methods described herein include animal or mammalian (e.g., human, fish, bovine, porcine, equine, monkey, ape, rat, murine, rabbit, ovine, insect, etc.) derived supplements, ingredients, or products. These sera and other media supplements are available commercially. Alternatively, sera and other media supplements described herein may be isolated from their natural sources or produced recombinantly by art-known methods that will be routine to one of ordinary skill. See Freshney, R. I., Culture of Animal Cells, New York: Alan R. Liss, Inc., pp. 74-78 (1983), and references cited therein; see also Harlow, E., and Lane, D., Antibodies: A Laboratory Manual, Cold Spring Harbor Laboratory, N.Y, 116-120 (1988).

Components that are often present in the final formulation in μg/mL or even μg/L amounts have typically been left out of standard powdered media due to homogeneity and/or stability concerns, and instead are typically added to the reconstituted 1× media as a concentrate, thereby increasing storage costs and causing production of a finished culture medium to become more costly and less efficient. In one aspect, such low-level components may be added to the base powder by first making a concentrate of the components and then spraying them into a portion of the powdered media that would be granulated with the concentrate. This would then be milled to a particle size in the same general size range as that of the bulk for blending. The ability to spray-in components in small amounts may be especially helpful in developing media that include trace elements, vitamins, viral inhibitors, growth factors, cytokines, and the like. Specifically, among others, the components to be added to a powdered medium include but are not limited to vitamins comprising retinol (A), thiamine (B1), riboflavin (B2), niacinamide (B3), pantothenic acid (B5), pyridoxamine (B6), biotin (B7), folic acid (B9) cobalamin (B12), ascorbic acid (C), cholecalciferol (D), tocopherol (E), phylloquinone (K), choline, inositol, lipoic acid, para-aminobenzoic acid, salts thereof, and trace elements including iron, manganese, copper, iodine, zinc, cobalt, fluoride, chromium, molybdenum, selenium, nickel, silicon, vanadium, salts thereof, or combinations thereof. Additional components to be added in low amounts to the culture media described herein may include, for example, growth factors (e.g., EGF, αFGF, βFGF, KGF, HGF, IGF-1, IGF-2, NGF, insulin, and the like), interleukins, colony-stimulating factors, interferons, attachment factors, extracellular matrix components s (e.g., collagens, laminins, proteoglycans, glycosaminoglycans, fibronectin, vitronectin, and the like), lipids (such as phospholipids, cholesterol, bovine cholesterol concentrate, fatty acids, sphingolipids and the like); extracts of animal tissues, glands or organs; antibiotics such as Geneticin carbenicillin, cefotaxime, anti-PPLO, Fungizone, hygromycin, kanamycin, neomycin, nystatin, penicillin, or streptomycin, etc.; and viral inhibitors (e.g., protease inhibitors, nucleoside analogues, and the like, which are known in the art).

Examples of buffers that may be prepared as described herein and/or that may be included in the culture media include, but are not limited to, buffered saline solutions, phosphate-buffered saline (PBS) formulations, Tris-buffered saline (TBS) formulations, HEPES-buffered saline (HBS) formulations, Hanks' Balanced Salt Solutions (HBSS), Dulbecco's PBS (DPBS), Earle's Balanced Salt Solutions, Puck's Saline Solutions, Murashige and Skoog Plant Basal Salt Solutions, Keller's Marine Plant Basal Salt Solutions, Provasoli's Marine Plant Basal Salt Solutions, and Kao and Michayluk's Basal Salt Solutions, and the like. Formulations for these buffers, which are commercially available, as well as for many other commonly used buffers, are well-known in the art and may be found for example in the Thermo Fisher Scientific Catalog, in the DIFCO & BBL Manual, 2n ed. (Becton, Dickinson and Company, 2009), and in the Millipore Sigma Cell Culture Catalogues.

Clinical solutions, particularly those used for parenteral nutrition, electrolyte balance or intravenous (IV) solutions are also described. Such clinical solutions include but are not limited to Ringer's, Ringer's lactate, 5% by mass dextrose in water, normal saline (0.9% by mass NaCl), hypotonic saline (0.45% by mass NaCl), 5% by mass dextrose in saline, and the like. Clinical solutions may further comprise one or more pharmaceutical compositions or components thereof.

### Methods of Preparing Compositions and Formulations

Also described is a method for preparing nutritive media, media supplements, media subgroups, buffers, or samples that contain a desired or effective amount or concentration of an ingredient(s), wherein at least one ingredient such as a carbohydrate or sugar (e.g., glucose) vitamins, an amino acid, a salt, a trace element, a growth factor and/or an amine (e.g., ethanolamine, spermine, spermidine, putrescene or para-aminobenzoic acid) is inputted into the manufacturing process at a higher amount as compared to the final product. The method of compensating for a loss or decrease in effective concentration of at least one ingredient during an agglomeration process comprising calculating or determining the amount of the ingredient to be added to the process as described herein to result in the final desired or effective amount.

### Methods for Determining the Effective Concentration of a Compound

One method for determining the effective concentration of a compound (e.g., a vitamin) in a test culture medium is as follows. Using a vitamin for the purposes of illustration, a known concentration of the vitamin is serially diluted into a culture medium lacking the vitamin. A second set of serial dilutions are set-up where the test culture medium is serially diluted into a culture medium also lacking the vitamin. Cells that require the vitamin for growth are then added to both sets of serially diluted samples and cultured under appropriate conditions. After a period of time, cell replication is measured (e.g., by cell counting or by measuring optical density). The measurements of the known concentrations are graphed to form a standard curve, to which the measurements from the test culture medium dilutions are compared to determine the effective concentration of the vitamin in the test culture medium. Any number of similar assays may be used to determine the amount of a metabolite(s) in a sample, which are available for cellular metabolism.

### Methods for Sterilizing a Composition and Formulation

Another embodiment is a method for sterilizing the nutritive media, media supplements, media subgroups and buffers described herein, as well as for sterilizing powdered nutritive media, media supplements, media subgroups and buffers prepared by standard methods such as ball-milling. Also described are methods for sterilizing or substantially sterilizing the samples including nutritive media, media supplements, media subgroups, and buffers described herein. Such additional methods may include filtration, heat sterilization, irradiation, or other chemical or physical methods. Nutritive media, media supplements, media subgroups, or buffers (prepared as described herein may be irradiated under conditions favoring sterilization. Since nutritive media, media supplements, media subgroups, and buffers are usually prepared in large volume solutions and frequently contain heat labile components, they are not amenable to sterilization by irradiation or by heating. Thus, nutritive media, media supplements, media subgroups, and buffers are commonly sterilized by contaminant-removal methods such as filtration which significantly increases the expense and time required to manufacture such media, media supplements, media subgroups, and buffers.

Nutritive media, media supplements, media subgroups, or buffers prepared according to the methods described herein can be sterilized by less expensive and more efficient methods. For example, powdered nutritive media, media supplements, media subgroups, or buffers may be irradiated under conditions favoring sterilization of these powders. Preferably, this irradiation is accomplished in bulk (i.e., following packaging of the sample, nutritive media, media supplement, media subgroup, or buffer), and most preferably this irradiation is accomplished by exposure of the bulk packaged sample, media, media supplement, media subgroup, or buffer described herein to a source of gamma rays under conditions such that bacteria, fungi, spores or viruses that may be resident in the powdered sample media, media supplements, media subgroups, or buffers are inactivated (i.e., prevented from replicating). Alternatively, irradiation may be accomplished by exposure of the sample, powdered media, media supplement, media subgroup, or buffer, prior to packaging, to a source of gamma rays or a source of ultraviolet light. The sample, media, media supplements, media subgroups and buffers described herein may alternatively be sterilized by heat treatment (if the subgroups, or components of the sample, nutritive media, media supplement, media subgroup, or buffer are heat stable), for example by flash pasteurization or autoclaving. As will be understood by one of ordinary skill in the art, the dose of irradiation or heat, and the time of exposure, required for sterilization will depend upon the bulk of the materials to be sterilized, and can easily be determined by the ordinarily skilled artisan without undue experimentation using art-known techniques, such as those described herein.

In one embodiment described herein, the bulk sample (e.g., nutritive media, media supplements, media subgroups, or buffers) (which are preferably in powdered form) are exposed to a source of irradiation (e.g., y (gamma) radiation) at a total dosage of about 10-100 kilograys (kGy), preferably a total dosage of about 15-75 kGy, 15-50 kGy, 15-40 kGy, 20-40 kGy or 25-45 kGy, more preferably a total dosage of about 20-30 kGy, and most preferably a total dosage of about 25-35 kGy, for about 1 hour to about 7 days, more preferably about 1 hour to about 5 days, 1 hour to about 3 days, about 1-24 hours or about 1-5 hours, and most preferably about 1-3 hours (“normal dose rate”). Alternatively, the bulk powders described herein may be sterilized at a “slow dose rate” of a total cumulative dosage of about 25-100 kGy over a period of about 1-5 days. During irradiation, the nutritive media, media supplements, media subgroups, or buffers (which are preferably in powdered form) are preferably stored at a temperature of about −70° C. to about room temperature (about 20-25° C.), most preferably at about −70° C. One of ordinary skill will appreciate, of course, that radiation dose and exposure times may be adjusted depending upon the bulk and/or mass of material to be irradiated; typical optimal irradiation dosages, exposure times and storage temperatures required for sterilization of powdered materials by irradiation or heat treatment are known in the art.

Following sterilization, unpackaged nutritive media, media supplements, media subgroups and buffers may be packaged under aseptic conditions, for example by packaging into containers such as sterile tubes, vials, bottles, bags, pouches, boxes, cartons, drums, and the like, in vacuum packaging, or integrated powder/solvent packaging described herein. Sterile packaged samples such as media, media supplements, media subgroups, and buffers may then be stored for extended periods of time as described herein.

### Methods of Reconstitution of Compositions and Formulations

The nutritive media, media supplements, media subgroups, and buffers described herein are readily soluble in a rehydrating solvent and are substantially dust free. For use, the media, media supplement, media subgroup, or buffer may be “rehydrated” or “reconstituted” in a volume of a solvent sufficient to produce the desired nutrient, electrolyte, ionic and pH conditions required for the particular use of the solvated media, media supplement, media subgroup, or buffer. This reconstitution is particularly facilitated because the media, media supplements, media subgroups, and buffers will readily dissolve and will produce little if any dust or insoluble material, unlike powdered nutritive media, media supplements, media subgroups, or buffers.

Solvents for use in reconstituting the nutritive media, media supplements, media subgroups, buffers, or samples described herein include, but are not limited to, the solvents described herein such as water, such as distilled and/or deionized water, serum (bovine or human serum and most particularly fetal bovine serum or calf serum), organic solvents (dimethylsulfoxide, acetone, ethanol and the like), or any combination thereof, any of which may contain one or more additional components (e.g., salts, polysaccharides, ions, detergents, stabilizers, etc.). For example, media supplements (such as animal sera) and buffers are preferably reconstituted in water to a 1× final concentration, or optionally to a higher concentration (e.g., 2×, 2.5×, 5×, 10×, 20×, 25×, 50×, 100×, 500×, 1000×, etc.) for the preparation of stock solutions or for storage. Alternatively, culture media may be reconstituted in a solution of media supplements (e.g., sera such as FBS) in water, such as those solutions wherein the media supplement is present at a concentration, for example, of 0.5%, 1%, 2%, 2.5%, 5%, 7.5%, 10%, 15%, 20%, 25%, 50%, or higher, by volume (vol/vol) in the water.

Reconstitution of the sample (e.g., nutritive media, media supplements, media subgroups, or buffers) is typically accomplished under aseptic conditions to maintain the sterility of the reconstituted sample, although the reconstituted sample may be sterilized by filtration or other sterilization methods that are well known in the art, following rehydration. Following their reconstitution, media, media supplements, media subgroups and buffers or other samples should be stored at temperatures below about 10° C., preferably at about 1-4° C., until use.

The reconstituted nutritive media, media supplements, media subgroups and buffers may be used to culture or manipulate cells according to standard cell culture techniques that are known to one of ordinary skill in the art. In such techniques, the cells to be cultured are contacted with the reconstituted media, media supplement, media subgroup, or buffer described herein under conditions favoring the cultivation or manipulation of the cells (such as controlled temperature, humidity, lighting, and atmospheric conditions). Cells that are particularly amenable to cultivation by such methods include, but are not limited to, bacterial cells, fish cells, yeast cells, plant cells, and animal cells. Such bacterial cells, yeast cells, plant cells and animal cells are available commercially from known culture depositories, e.g., the American Type Culture Collection (Manassas, Va.) and others that will be familiar to one of ordinary skill in the art. Preferred animal cells for cultivation by these methods include, but are not limited to, insect cells (most preferably Drosophila cells, Spodoptera cells and Trichoplusia cells), nematode cells (most preferably C. elegans cells) and mammalian cells (most preferably CHO cells, COS cells, VERO cells, BHK cells, AE-1 cells, SP2/0 cells, L5.1 cells, hybridoma cells and human cells, such as 293 cells, PER-C6 cells and Hela cells), any of which may be a somatic cell, a germ cell, a normal cell, a diseased cell, a transformed cell, a mutant cell, a stem cell, a precursor cell or an embryonic cell, embryonic stem cells (ES cells), cells used for virus or vector production (i.e., 293, PerC 6), cells derived from primary human sites used for cell or gene therapy, i.e., lymphocytes, hematopoietic cells, other white blood cells (WBC), macrophage, neutrophils, dendritic cells, and any of which may be an anchorage-dependent or anchorage-independent (i.e., “suspension”) cell. Another aspect is the manipulation or cultivation of cells and/or tissues for tissue or organ transplantation or engineering, i.e., hepatocyte, pancreatic islets, osteoblasts, osteoclasts/chondrocytes, dermal or muscle or other connective tissue, epithelial cells, tissues like keratinocytes, cells of neural origin, cornea, skin, organs, and cells used as vaccines, i.e., blood cells, hematopoietic cells other stem cells or progenitor cells, and inactivated or modified tumor cells of various histotypes.

### Methods of Manipulating or Culturing Cells

Another embodiment is a method of manipulating or culturing one or more cells comprising contacting said cells with the cell culture reagents described herein, particularly reconstituted nutritive media, media supplement, media subgroup, or buffer and incubating said cell or cells under conditions favoring the cultivation or manipulation of the cell or cells. Any cell may be cultured or manipulated according to the present methods, particularly bacterial cells, yeast cells, plant cells, animal cells and other cells or cell lines described herein. Cells cultured or manipulated according to this aspect described herein may be normal cells, diseased cells, transformed cells, mutant cells, somatic cells, germ cells, stem cells, precursor cells or embryonic cells, any of which may be established cell lines or obtained from natural sources.

Nutritive media, media supplements and media subgroups produced by the present methods are any media, media supplement or media subgroup (serum-free or serum-containing) which may be used to manipulate or support the growth of a cell, which may be a bacterial cell, a fungal cell (particularly a yeast cell), a plant cell, or an animal cell (particularly an insect cell, a nematode cell, or a mammalian cell, most preferably a human cell), any of which may be a somatic cell, a germ cell, a normal cell, a diseased cell, a transformed cell, a mutant cell, a stem cell, a precursor cell, or an embryonic cell. Preferred such nutritive media include, but are not limited to, cell culture media, most preferably a bacterial cell culture medium, plant cell culture medium or animal cell culture medium. Preferred media supplements include, but are not limited to, undefined supplements such as extracts or hydrolysates of bacterial, animal or plant cells, glands, tissues or organs (particularly bovine pituitary extract, bovine brain extract and chick embryo extract); and biological fluids or blood derived products (particularly animal sera, and most preferably bovine serum (particularly fetal bovine, newborn calf, or normal calf serum), horse serum, porcine serum, rat serum, murine serum, rabbit serum, monkey serum, ape serum, or human serum, any of which may be fetal serum) and extracts thereof (more preferably serum albumin and most preferably bovine serum albumin or human serum albumin). Medium supplements may also include defined replacements such as StemPro LipoMAX, OptiMAb, Knock-Out Serum Replacement (Gibco, Thermo Fisher Scientific Inc.), and the like, which can be used as substitutes for the undefined media supplements described above. Such supplements may also comprise defined components, including but not limited to, hormones, cytokines, neurotransmitters, lipids, attachment factors, proteins, amino acids, and the like.

The media described herein, upon being reconstituted with a solvent, can be used for the growth and/or cultivation of organisms such as, e.g., filamentous fungi, transgenic plants (e.g., tobacco, rice, and Lemna), lichens, or algae, or cells derived from any of the aforementioned organisms.

### Forms of Media Supplements or Feeds

Also described are forms of media supplements or feeds. In one aspect, the supplement includes one or more amino acids. In another aspect, a salt of an amino acid is used. In another aspect, the salt is a sodium salt. In another aspect, monobasic and dibasic phosphate salts are used. A preferred cation is sodium. In another aspect, the monobasic and dibasic salts are provided such that a resultant pH, for example, about 8 pH is obtained. Depending on the formulation, while the ratio of monobasic to dibasic salts may be dictated by desired pH, different total salt concentrations should be tried to optimize solubility, especially when concentrated or highly concentrated supplements are to be used. The pH can also be confirmed when assessing the salt concentration. When an amino acid is not provided as a salt, preferably the pH effect of the acid is countered by a tribasic phosphate, preferably a sodium tribasic phosphate. While sodium is preferred as a cation, other metals, such as potassium, calcium, magnesium may be used. If a specific counter ion is desired, it may be available as a phosphate salt. In another aspect, the supplement powder dissolves rapidly. In another aspect, the supplement can be prepared and used as a highly concentrated mixture, for example, with one or more components at a concentration about 2× or more, preferably 3×, 5×, 8×, 10×, 12×, 15×, 20×, 25×, 50×, 75×, 85×, 95×, or even about 100× or more times the concentration of that component in the medium being supplemented. The concentration of each desired ingredient of the supplement can be independently selected. In another aspect, the supplement is prepared by reconstituting with water under sterile conditions. In another aspect, the supplement is sterilized by filtration.

A supplement may have no ingredients in common with the medium being supplemented or may have one or more ingredients in common. The supplement may differ from the medium being supplemented in at least one manner, such as a different concentration of one or more ingredients, for example a different ratio of two ingredients, a different ingredient mix, additional ingredients, or omitted ingredients in the supplement. For example, a supplement may omit salts to the extent feasible and may contain, for example, significantly enhanced concentrations of growth factors or amino acids. A preferred supplement formulation contains at least 2, more preferably 3, but perhaps at least 4, 5, 6, 7, 8, 9, 10, 11, 12, 13, 14, 15, 17, 20, or more amino acids including salts, or dimers thereof.

In some embodiments, feed supplements as described herein are utilized to supplement a medium that has or is being used to culture cells, e.g., as the cells are cultured, some ingredients are removed from the medium by the cells. In some embodiments described herein, the feed supplement is used, inter alia, to replace some or all of these ingredients. In some embodiments, the supplement contains the majority of the ingredients that were in the original medium to be supplemented, but the feed medium is lacking at least one ingredient. In some embodiments, the feed supplement is lacking 1, 2, 3, 4, 5, 6, 7, 8, 9, 10, 11, 12, 13, 14, 15, or more ingredients as compared to the concentration in the original culture medium being supplemented. In some embodiments, the feed supplement is added in a concentrated form, e.g., at 2×, 3×, 4×, 5×, 6×, 7×, 8×, 9×, 10×, 15×, 20×, 30×, 40×, 50×, 100×, 200×, 300×, 400×, 500× or 1000×. By concentrated form is meant that at least one of the ingredients in the feed supplement is at a concentration higher than what is the desired concentration in the culture medium. In some embodiments, ingredients for a feed supplement may be divided into multiple feed supplement media, e.g., based upon compatible subgroups.

In another embodiment, the media, supplement, feed, subgroup, or buffer is added to a culture to optimize the concentration of a particular component, supplement a depleted or omitted component, or replace a component that has been consumed or degraded by the culture. The media can be dissolved in a solvent prior to addition to the culture or added in solid form.

In another embodiment, a cell culture is analyzed to determine the concentration of one or more media components. Typical analysis methods can be used to determine the concentration of a component such as HPLC, mass spectrometry, ELISA, standard curve assays, and other methods known in the art. If the concentration of a component is less than a desired range, a supplement may be added to the culture to increase the concentration to the desired range. The media can be dissolved in a solvent prior to addition to the culture or added and dissolved in situ.

In some embodiments, a portion of the ingredients of a feed supplement is reconstituted from an agglomerated supplement. In some embodiments, additional ingredients are added to the reconstituted media or a liquid form of a supplement or feed. In some embodiments, the additional ingredients comprise amino acids or antibiotics.

### Osmolality of a Composition and Formulation

Osmolality (a measure of osmotic pressure) of cell culture medium is important as it helps regulate the flow of substances in and out of the cell. It is typically controlled by the addition or subtraction of salt in a culture medium. Rapid increases in osmolality (e.g., addition of concentrated feed supplement with elevated osmolality relative to the base growth medium) may result in stressed, damaged, or dead cells. Maintaining an optimal osmolality range during cell culture/growth is desirable for cell function and/or bioproduction success.

Base growth medium osmolality ranges from 250 mOsmo/kg to 350 mOsmo/kg. In some embodiments, addition of a concentrated feed supplement described herein increases osmolality by about 25 mOsmo/kg or by between from about 0 to about 100, about 0.01 to about 100, about 0.1 to about 100, about 1 to about 100, about 10 to about 100, about 50 to about 100, about 75 to about 100, about 1 to about 10, about 1 to about 50, about 1 to about 75, about 10 to about 50, about 15 to about 35, about 25 to about 50, or about 20 to about 30 mOsmo/kg. In some embodiments, the osmolality of a concentrated feed supplement medium described herein (e.g., a 5× concentrated feed supplement medium) has an osmolality between from about 0 to about 1500; 1 to about 1000; 1 to about 750; 1 to about 500; 1 to about 400; 1 to about 300; 1 to about 200; 1 to about 100; 1 to about 50; 50 to about 1000; 100 to about 1000; 300 to about 1000; 500 to about 1000; 750 to about 1000; 100 to about 200; 200 to about 300; 300 to about 400; 400 to about 500; 450 to about 500; 500 to about 600; 550 to about 650; 600 to about 700; 750 to about 850; 700 to about 800; 800 to about 900; 900 to about 1000; 1000 to about 1250; or about 1250 to about 1500 mOsmo/kg. In some embodiments, the osmolality of a concentrated feed supplement medium described herein is between from about 3.0× to about 3.5×, about 3.5× to about 4.5×, about 4.5× to about 5.5×, about 5.5× to about 6.5×, about 6.5× to about 7.5×, about 7.5× to about 8.5×, about 8.5× to about 9.5×, about 9.5× to about 10.5×, about 10.5× to about 11.5×, about 11.5× to about 12.5×, about 12.5× to about 13.5×, about 13.5× to about 14.5×, about 14.5× to 18.5 to about 19.5×, about 19.5× to about 20.5×, about 3× to about 10×, about 5× to about 10×, about 10× to about 15×, about 15× to about 20×, about 20× to about 25×, or about 25× to about 100× as compared to the osmolality of the medium being supplemented or fed.

### Methods for Preparation at Reduced Cost

Described herein are methods for the preparation of nutritive media, media supplements, media subgroups, buffers, and cells at reduced cost. The compositions and methods provide for the preparation of nutritive media, media supplements, media subgroups, buffers, and cells at reduced cost and reduced inconvenience. The cost reductions are due to the several factors. For example, the media, media supplement, media subgroup, and buffer formulations may be produced with smaller production facilities since the large stir tanks required for 1× formulations are not required. In addition, the media, media supplement, media subgroup and buffer formulations may be prepared on an as needed basis using “just in time” production techniques, which reduce inventory, storage, and labor costs. The time required for the preparation and shipping of the media, media supplement, media subgroup and buffer formulations may be reduced from 6-8 weeks to as little as one day. The automatically pH-adjusting media described herein also provide significant cost, time savings, and reduce the tendency for introduction of contamination into reconstituted media that may occur during the pH adjustment process according to methods using traditional dry powder or bulk liquid media.

### Preparation of Large Quantities of a Composition and Formulation

Also described is the preparation of nutritive media, media supplements, media subgroups, or buffers which may be used to prepare very large quantities of 1× media, media supplements, media subgroups, or buffers (e.g., 100,000 liters or more) which would require only one quality control test compared to multiple quality control tests for multiple batches produced according to other commonly used techniques. Importantly, the media, media supplement, media subgroup, or buffer formulations are more consistent between batches since the individual components are more stable. Further, the media, media supplement, media subgroup, or buffer formulations can easily be dispensed to produce a particular volume without having to weigh the dry component.

### Methods of Producing Agglomerated Powders

Another embodiment described herein is a method for producing a form of an agglomerated nutritive medium powder, an agglomerated medium supplement powder, an agglomerated nutritive medium subgroup powder, or an agglomerated buffer powder, said method comprising agglomerating a nutritive medium powder, medium supplement powder, nutritive medium subgroup powder, or buffer powder, with a solvent comprising at least one lipid dissolved therein, said solvent delivering said at least one lipid for incorporation in said nutritive medium powder, medium supplement powder, nutritive medium subgroup powder, or buffer powder. In one aspect, the agglomerating comprises fluid bed agglomeration.

### Kits

The media, media supplements, media subgroups, buffers, cells, and cell-containing compositions described herein are ideally suited for preparation of kits. Such a kit may comprise one or more containers such as vials, test tubes, bottles, packages, pouches, drums, and the like. Each of the containers may contain one or more of the cell culture reagents, nutritive media, media supplements, media subgroups, cells, or buffers described herein, or combinations thereof. Such cell culture reagents, nutritive media, media supplements, media subgroups, buffers or cells may be hydrated or dehydrated but are typically dehydrated preparations produced by the methods described herein. Such preparations may be sterile or substantially sterile.

A first container may contain, for example, a nutritive media, media supplement, media subgroup, or a buffer described herein, or any component or subgroup thereof, such as any of those nutritive media, media supplements, media subgroups, or buffers described herein that are described herein. Additional dry, or liquid nutritive media, buffers, extracts, supplements, components, or subgroups may be contained in additional containers in the present kits. The kits may also contain, in one or more additional containers, one or more cells such as bacterial cells, yeast cells, plant cells, or animal cells. Such cells may be lyophilized, dried, frozen, or otherwise preserved, or may be spray-dried according to the methods described herein or treated by the method described herein. In addition, the kits described herein may further comprise one or more additional containers, containing, for example, I-glutamine, optionally complexed with one or more divalent cations. The kits may further comprise one or more additional containers containing a solvent to be used in reconstituting the dry powder pharmaceutical, or clinical compositions, cell culture reagents, nutritive media, media supplements, media subgroups and/or buffers; such solvents may be aqueous or organic and include buffer solutions, saline solutions, nutritive medium solutions, nutritive medium supplement solutions including sera such as bovine sera, fetal bovine sera, calf sera, human sera, or combinations thereof. Other ingredients that are not compatible for admixture with the nutritive media, buffers, pharmaceutical compositions, extracts, supplements, components, or subgroups described herein may be contained in one or more additional containers to avoid mixing of incompatible components. An exemplary kit may comprise a container containing media or reconstitution optionally of a volume sufficient to contain the reconstituting solvent, instructions for reconstitution and means for accessing the media such as a tear strip or a port for introducing the reconstituting solvent.

The number and types of containers contained in a given kit (e.g., for making a nutritive medium, medium supplement, medium subgroup, or buffer) may vary depending on the desired product or the type of pharmaceutical or clinical compositions, media, media supplement, media subgroup, or buffer to be prepared. Typically, the kit will contain the respective containers containing the components or supplements necessary to make a particular pharmaceutical or clinical composition, media, media supplement, media subgroup, or buffer. However, additional containers may be included in the kit described herein so that different pharmaceutical or clinical compositions, media, media supplements, media subgroups, or buffers can be prepared by mixing different amounts of various components, supplements, subgroups, buffers, solvents, etc., to make different pharmaceutical or clinical compositions, media, media supplement, media subgroup, or buffer formulations. The containers may be sealable, re-sealable, tamper-resistant, tamper evident, or open-indicating. The containers may have multiple modes, such as a resealable bag within a tamper-resistant or open-evident sealed pail.

Kits may also contain one or more of labels, manufacturing dates, expiration dates, use by dates, packaging, branding, instructions for use, material safety data sheets (MSDS), hazard or warning placards, or product literature.

### EMBODIMENTS

One embodiment described herein is a room-temperature-stable cell culture media or supplement composition comprising: a base cell culture medium comprising one or more amino acids, salts, buffers, trace minerals, lipids, nucleic acids, or proteins; one or more carbohydrates; and one or more vitamins; wherein the carbohydrates and vitamins are segregated from the base cell culture medium. In one aspect, the composition is stable at a temperature from about 15° C. to about 30° C. In another aspect, the composition is stable at room temperature for about 1 day to about 2 years. In another aspect, the segregation comprises one or more of: physical separation of the base cell culture medium from the carbohydrates and/or vitamins in different containers; separation of the base cell culture medium from the carbohydrates and/or vitamins in the same container; granulation or agglomeration of the base cell culture medium; powdering, granulation, or agglomeration of the carbohydrates; powdering, granulation, agglomeration, encapsulation, or coating of the vitamins; or combinations thereof. In another aspect, the carbohydrates and/or vitamins are intermixed with the base cell culture medium to form the temperature-stable cell culture media or supplement composition. In another aspect, the base cell culture medium is granulated or agglomerated and situated in a first container; the carbohydrates are granulated or agglomerated and situated in a second container; and the vitamins are situated in a third container. In another aspect, the base cell culture medium is granulated or agglomerated and situated in a first container; the carbohydrates are powdered and situated in a second container; and the vitamins are situated in a third container. In another aspect, the base cell culture medium and the carbohydrates are separately granulated or agglomerated, admixed, and situated in a first container; and the vitamins are situated in a second container. In another aspect, the base cell culture medium is granulated or agglomerated, the carbohydrates are powdered and admixed with the agglomerated base cell culture medium, and situated in a first container; and the vitamins are situated in a second container. In another aspect, the base cell culture medium is granulated or agglomerated and situated in a first container; and the carbohydrates and the vitamins are admixed, granulated, or agglomerated, and situated in a second container. In one aspect, the carbohydrates and/or the vitamins are powdered or agglomerated, or a combination thereof. In another aspect, the base cell culture medium is granulated or agglomerated and situated in a first container; the carbohydrates are granulated or agglomerated, admixed with the granulated or agglomerated base cell culture medium and situated in the first container; the vitamins are situated in a third container. In another aspect, the base cell culture medium is granulated or agglomerated and situated in a first container; the carbohydrates are powdered, admixed with the granulated or agglomerated base cell culture medium and situated in the first container; the vitamins are situated in a third container; the vitamins may be powered, agglomerated, or in liquid form. In another aspect, the interaction or chemical reaction of the carbohydrates and/or vitamins with the base cell culture medium is inhibited or reduced owing to segregation in separate containers. In another aspect, the carbohydrates comprise one or more of glucose, fructose, or trehalose. In another aspect, the one or more vitamins comprise one or more of retinol (A), thiamine (B1), riboflavin (B2), niacinamide (B3), pantothenic acid (B5), pyridoxamine (B6), biotin (B7), folic acid (B9) cobalamin (B12), ascorbic acid (C), cholecalciferol (D), tocopherol (E), phylloquinone (K), choline, inositol, lipoic acid, or para-aminobenzoic acid.

Another embodiment described herein is a room-temperature-stable cell culture media composition comprising: granules or agglomerates of a base cell culture medium comprising one or more amino acids, salts, buffers, trace minerals, lipids, nucleic acids, or proteins; and granules or agglomerates of one or more carbohydrates. In one aspect, the granules or agglomerates of the base cell culture medium are admixed with the granules or agglomerates of one or more carbohydrates. In another aspect, the granules or agglomerates of the base cell culture medium are layered over the granules or agglomerates of one or more carbohydrates; or the granules or agglomerates of one or more carbohydrates are layered over the granules or agglomerates of the base cell culture medium.

Another embodiment described herein is a room-temperature-stable cell culture media composition comprising granules or agglomerates of one or more carbohydrates and one or more vitamins.

Another embodiment described herein is a room-temperature-stable cell culture media composition comprising: granules or agglomerates of a base cell culture medium comprising one or more amino acids, salts, buffers, trace minerals, lipids, nucleic acids, or proteins; and granules or agglomerates of one or more carbohydrates and one or more vitamins. In one aspect, the granules or agglomerates of the base cell culture medium are admixed with the granules or agglomerates of one or more carbohydrates and one or more vitamins. In another aspect, the granules or agglomerates of the base cell culture medium are layered over the granules or agglomerates of one or more carbohydrates and one or more vitamins; or the granules or agglomerates of one or more carbohydrates and one or more vitamins are layered over the granules or agglomerates of the base cell culture medium.

Another embodiment described herein is a room-temperature-stable cell culture media composition comprising powders of one or more carbohydrates and one or more vitamins.

Another embodiment described herein is a room-temperature-stable cell culture media composition comprising: granules or agglomerates of a base cell culture medium comprising one or more amino acids, salts, buffers, trace minerals, lipids, nucleic acids, or proteins; and powders of one or more carbohydrates and one or more vitamins. In one aspect, the granules or agglomerates of the base cell culture medium are admixed with the powders of one or more carbohydrates and one or more vitamins. In another aspect, the granules or agglomerates of the base cell culture medium are layered over the powders of one or more carbohydrates and one or more vitamins; or the powders of one or more carbohydrates and one or more vitamins are layered over the granules or agglomerates of the base cell culture medium.

Another embodiment described herein is a room-temperature-stable cell culture media composition comprising: granules or agglomerates of a base cell culture medium comprising one or more amino acids, salts, buffers, trace minerals, lipids, nucleic acids, or proteins; and granules or agglomerates of one or more carbohydrates; and one or more encapsulated or coated vitamins. In one aspect, the granules or agglomerates of the base cell culture medium are admixed with the granules or agglomerates of one or more carbohydrates; and the encapsulated vitamins are admixed with the admixture of the base cell culture medium and carbohydrates or layer over or under the admixture of the base cell culture medium and carbohydrates. In another aspect, the vitamins are coated or incapsulated in one or more of alginate, agar, agarose, pectin, or guar gum.

Another embodiment described herein is a method of manufacturing the room-temperature-stable cell culture composition described herein, the method comprising: (a) combining the one or more amino acids, salts, buffers, trace minerals, lipids, nucleic acids, or proteins to form the base cell culture medium; (b) granulating or agglomerating the base cell culture medium; (c) combining the granulated or agglomerated the base cell culture medium with carbohydrates. In one aspect, the method further comprises preparing a solution concentrate of one or more vitamins. In another aspect, the method further comprises adding the vitamin solution concentrate to the combination of the base cell culture medium and carbohydrates upon solubilization. In another aspect, steps (c)-(d) comprises: (c) granulating or agglomerating carbohydrates and vitamins; and (d) combining the granulated or agglomerated base cell culture medium with the granulated or agglomerated carbohydrates and vitamins. In another aspect, steps (c)-(d) comprises: (c)(i) granulating or agglomerating the carbohydrates; and (c)(ii) encapsulating or coating the vitamins; (d) combining the granulated or agglomerated base cell culture medium with the granulated or agglomerated carbohydrates and the coated or encapsulated vitamins.

Another embodiment described herein is a method for making a liquid cell culture media, the method comprising solubilizing any of the compositions described herein with water.

In one aspect, the method further comprises contacting one or more mammalian cells with the liquid media and culturing the cells.

Another embodiment described herein is a kit for storing a room temperature-stable cell culture media comprising: (a) a base cell culture medium comprising one or more amino acids, salts, buffers, trace minerals, lipids, nucleic acids, or proteins, wherein the base cell culture medium is optionally granulated or agglomerated; (b) one or more carbohydrates, wherein the carbohydrates are optionally granulated or agglomerated; (c) one or more vitamins; wherein the vitamins are optionally granulated, agglomerated, encapsulated, coated, powdered, or in a solution concentrate; and (d) one or more containers comprising drums, buckets, boxes, bags, pouches, or combinations thereof. In one aspect, the one or more carbohydrates are powdered. In another aspect, the base cell culture medium comprises granules or agglomerates situated in a first container; the carbohydrates comprise separate granules or agglomerates situated in a second container; and the vitamins comprise a solution concentrate situated in a third container. In another aspect, the base cell culture medium comprises granules or agglomerates, and the carbohydrates comprise separate granules or agglomerates, both of which are mixed and situated in a first container; and the vitamins comprise a solution concentrate situated in a second container. In another aspect, the base cell culture medium comprises granules or agglomerates, the carbohydrates and vitamins are combined in mixed granules or agglomerates, each of which are present as an intermixed composition situated within a single container. In another aspect, the base cell culture medium comprises granules or agglomerates, and the carbohydrates comprise separate granules or agglomerates, both of which are situated in a first container, wherein the cell culture medium granules or agglomerates is layered over the carbohydrate granules or agglomerates or the carbohydrates granules or agglomerates is layered over the cell culture medium granules or agglomerates; and the vitamins comprise a solution concentrate situated in a second container. In another aspect, the base cell culture medium comprises granules or agglomerates; the carbohydrates and vitamins are combined in mixed granules or agglomerates; each of which are present as an intermixed composition situated within a single container. In another aspect, the base cell culture medium comprises granules or agglomerates; the carbohydrates comprise separate granules or agglomerates; and the vitamins comprise encapsulated particles; each of which are present as an intermixed composition situated within a single container. In another aspect, the interaction or chemical reaction of the carbohydrates and/or vitamins with the base cell culture medium is inhibited or reduced. In another aspect, the kit optionally further comprises one or more of labels, manufacturing dates, expiration dates, use by dates, packaging, branding, instructions for use, material safety data sheets (MSDS), hazard or warning placards, or product literature.

Another embodiment described herein is a method of making a cell culture media using any of the kits described herein, the method comprises combining the kit components (a)-(c) and solubilizing the components with water, forming a liquid medium. In one aspect, the method further comprises contacting one or more mammalian cells with the liquid medium and culturing the cells.

Another embodiment described herein is the use of any of the compositions or the kits for cell culture media. In one aspect, the media is used for culturing a mammalian cell in vitro. In another aspect, the mammalian cell comprises CHO cells or HEK cells.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### CLAUSES

Various embodiments and aspects of the inventions described herein are summarized by the following clauses:

Clause 1. A room temperature-stable cell culture media or supplement composition comprising:
a base cell culture medium comprising one or more amino acids, salts, buffers, trace minerals, lipids, nucleic acids, or proteins; one or more carbohydrates; and one or more vitamins;

wherein the carbohydrates and vitamins are segregated from the base cell culture medium.

Clause 2. The composition of clause 1, wherein the composition is stable at a temperature from about 15° C. to about 30° C.

Clause 3. The composition of clause 1 or 2, wherein the composition is stable at room temperature for about 1 day to about 2 years.

Clause 4. The composition of any one of clauses 1-3, wherein the segregation comprises one or more of:
physical separation of the base cell culture medium from the carbohydrates and/or vitamins in different containers; separation of the base cell culture medium from the carbohydrates and/or vitamins in the same container;
granulation or agglomeration of the base cell culture medium;
granulation or agglomeration of the carbohydrates;
granulation, agglomeration, encapsulation, or coating of the vitamins; or combinations thereof.

Clause 5. The composition of clause 4, wherein the carbohydrates and/or vitamins are intermixed with the base cell culture medium to form the temperature-stable cell culture media or supplement composition.

Clause 6. The composition of clause 4, wherein the base cell culture medium is granulated or agglomerated and situated in a first container;
the carbohydrates are granulated or agglomerated and situated in a second container; and the vitamins are situated in a third container.

Clause 7. The composition of clause 4, wherein the base cell culture medium and the carbohydrates are separately granulated or agglomerated, admixed, and situated in a first container; and the vitamins are situated in a second container.

Clause 8. The composition of clause 4, wherein the base cell culture medium is granulated or agglomerated and situated in a first container; and the carbohydrates and the vitamins are admixed, granulated, or agglomerated, and situated in a second container.

Clause 9. The composition of clause 4, wherein the base cell culture medium is granulated or agglomerated and situated in a first container;
the carbohydrates are granulated or agglomerated, admixed with the granulated or agglomerated base cell culture medium and situated in the first container;
the vitamins are situated in a third container.

Clause 10. The composition of any one of clauses 1-10, wherein the interaction or chemical reaction of the carbohydrates and/or vitamins with the base cell culture medium is inhibited or reduced owing to segregation in separate containers.

Clause 11. The composition of any one of clauses 1-11, wherein the carbohydrates comprise one or more of glucose, fructose, or trehalose.

Clause 12. The composition of any one of clauses 1-12, wherein the one or more vitamins comprise one or more of retinol (A), thiamine (B1), riboflavin (B2), niacinamide (B3), pantothenic acid (B5), pyridoxamine (B6), biotin (B7), folic acid (B9) cobalamin (B12), ascorbic acid (C), cholecalciferol (D), tocopherol (E), phylloquinone (K), choline, inositol, lipoic acid, or para-aminobenzoic acid.

Clause 13. A room temperature-stable cell culture media composition comprising:
granules or agglomerates of a base cell culture medium comprising one or more amino acids, salts, buffers, trace minerals, lipids, nucleic acids, or proteins; and
granules or agglomerates of one or more carbohydrates.

Clause 14. The composition of clause 13, wherein the granules or agglomerates of the base cell culture medium are admixed with the granules or agglomerates of one or more carbohydrates.

Clause 15. The composition of clause 13, wherein:
the granules or agglomerates of the base cell culture medium are layered over the granules or agglomerates of one or more carbohydrates; or the granules or agglomerates of one or more carbohydrates are layered over the granules or agglomerates of the base cell culture medium.

Clause 16. A room temperature-stable cell culture media composition comprising granules or agglomerates of one or more carbohydrates and one or more vitamins.

Clause 17. A room temperature-stable cell culture media composition comprising:
granules or agglomerates of a base cell culture medium comprising one or more amino acids, salts, buffers, trace minerals, lipids, nucleic acids, or proteins; and granules or agglomerates of one or more carbohydrates and one or more vitamins.

Clause 18. The composition of clause 17, wherein the granules or agglomerates of the base cell culture medium are admixed with the granules or agglomerates of one or more carbohydrates and one or more vitamins.

Clause 19. The composition of clause 17, wherein:
the granules or agglomerates of the base cell culture medium are layered over the granules or agglomerates of one or more carbohydrates and one or more vitamins; or the granules or agglomerates of one or more carbohydrates and one or more vitamins are layered over the granules or agglomerates of the base cell culture medium.

Clause 20. A room temperature-stable cell culture media composition comprising:
granules or agglomerates of a base cell culture medium comprising one or more amino acids, salts, buffers, trace minerals, lipids, nucleic acids, or proteins; and granules or agglomerates of one or more carbohydrates; and one or more encapsulated or coated vitamins.

Clause 21. The composition of clause 20, wherein the granules or agglomerates of the base cell culture medium are admixed with the granules or agglomerates of one or more carbohydrates; and the encapsulated vitamins are admixed with the admixture of the base cell culture medium and carbohydrates or layer over or under the admixture of the base cell culture medium and carbohydrates.

Clause 22. The composition of clause 20, wherein the vitamins are coated or incapsulated in one or more of alginate, agar, agarose, pectin, or guar gum.

Clause 23. A method of manufacturing the room temperature-stable cell culture composition of any one of clauses 1-19, the method comprising:
(a) combining the one or more amino acids, salts, buffers, trace minerals, lipids, nucleic acids, or proteins to form the base cell culture medium;
(b) granulating or agglomerating the base cell culture medium;
(c) combining the granulated or agglomerated the base cell culture medium with carbohydrates.

Clause 24. The method of clause 23, wherein the method further comprises preparing a solution concentrate of one or more vitamins.

Clause 25. The method of clause 24, wherein the method further comprises adding the vitamin solution concentrate to the combination of the base cell culture medium and carbohydrates upon solubilization.

Clause 26. The method of clause 23, wherein steps (c)-(d) comprises:
(c) granulating or agglomerating carbohydrates and vitamins; and
(d) combining the granulated or agglomerated base cell culture medium with the granulated or agglomerated carbohydrates and vitamins.

Clause 27. The method of clause 23, wherein steps (c)-(d) comprises:
(c)(i) granulating or agglomerating the carbohydrates; and
(c)(ii) encapsulating or coating the vitamins;
(d) combining the granulated or agglomerated base cell culture medium with the granulated or agglomerated carbohydrates and the coated or encapsulated vitamins.

Clause 28. A method for making a liquid cell culture media, the method comprising solubilizing the composition of any one of clauses 1-22 with water.

Clause 29. The method of clause 28, further comprising contacting one or more mammalian cells with the liquid media and culturing the cells.

Clause 30. A kit for storing a temperature stable cell culture media comprising:
(a) a base cell culture medium comprising one or more amino acids, salts, buffers, trace minerals, lipids, nucleic acids, or proteins, wherein the base cell culture medium is optionally granulated or agglomerated;
(b) one or more carbohydrates, wherein the carbohydrates are optionally granulated or agglomerated;
(c) one or more vitamins; wherein the vitamins are optionally granulated, agglomerated, encapsulated, coated, powdered, or in a solution concentrate; and
(d) one or more containers comprising drums, buckets, boxes, bags, pouches, or combinations thereof.

Clause 31. The kit of clause 30, wherein the base cell culture medium comprises granules or agglomerates situated in a first container; the carbohydrates comprise separate granules or agglomerates situated in a second container; and the vitamins comprise a solution concentrate situated in a third container.

Clause 32. The kit of clause 30, wherein the base cell culture medium comprises granules or agglomerates, and the carbohydrates comprise separate granules or agglomerates, both of which are mixed and situated in a first container; and the vitamins comprise a solution concentrate situated in a second container.

Clause 33. The kit of clause 30, wherein the base cell culture medium comprises granules or agglomerates, the carbohydrates and vitamins are combined in mixed granules or agglomerates, each of which are present as an intermixed composition situated within a single container.

Clause 34. The kit of clause 30, wherein the base cell culture medium comprises granules or agglomerates, and the carbohydrates comprise separate granules or agglomerates, both of which are situated in a first container, wherein the cell culture medium granules or agglomerates is layered over the carbohydrate granules or agglomerates or the carbohydrates granules or agglomerates is layered over the cell culture medium granules or agglomerates; and the vitamins comprise a solution concentrate situated in a second container.

Clause 35. The kit of clause 30, wherein the base cell culture medium comprises granules or agglomerates; the carbohydrates and vitamins are combined in mixed granules or agglomerates; each of which are present as an intermixed composition situated within a single container.

Clause 36. The kit of clause 30, wherein the base cell culture medium comprises granules or agglomerates; the carbohydrates comprise separate granules or agglomerates; and the vitamins comprise encapsulated particles; each of which are present as an intermixed composition situated within a single container.

Clause 37. The kit of clause 30, wherein the interaction or chemical reaction of the carbohydrates and/or vitamins with the base cell culture medium is inhibited or reduced.

Clause 38. The kit of any one of clauses 30-37, wherein the kit optionally further comprises one or more of labels, manufacturing dates, expiration dates, use by dates, packaging, branding, instructions for use, material safety data sheets (MSDS), hazard or warning placards, or product literature.

Clause 39. A method of making a cell culture media using any one of the kits of clauses 30-38, the method comprises combining the kit components (a)-(c) and solubilizing the components with water, forming a liquid medium.

Clause 40. The method of clause 39, further comprising contacting one or more mammalian cells with the liquid medium and culturing the cells.

Clause 41. Use of the compositions of any one of clauses 1-22, or the kits of any one of clauses 30-37 for cell culture media.

Clause 42. The use of clause 40, wherein the media is used for culturing a mammalian cell in vitro.

Clause 43. The use of clause 42, wherein the mammalian cell comprises CHO cells or HEK cells.

### EXAMPLES

### Example 1

### Stability Testing

An experiment was designed to evaluate the stability of a complete basal (DYNAMIS) and a cell feed (EFFICIENTFEED C+) AGT media with or without glucose at 37° C. Efficient Feed C+(EFC+) and DYNAMIS, GIBCO, Thermo Fisher Scientific) with and without glucose were incubated for up to 95 days at 37° C. in either a sealed container (Securitainer, Thermo Fisher Scientific) or in a vacuum sealed bag. The AGT media was then dispensed into containers and photographed. See FIG. 2A, B. The pictures indicated with an asterisk show significant appearance change consistent with Maillard-reaction-induced degradation. The media were analyzed by HPLC, and degradation of specific components were identified (data not shown). The media was also evaluated for cell viability as discussed in Example 2.

### Example 2

### Cell Assays

Before and after stability testing, DYNAMIS and EFFICIENTFEED C+(EFC+) (GIBCO, Thermo Fisher Scientific) media with and without glucose were reconstituted and supplemented with glucose (as needed) up to 6 g/L for DYNAMIS or 32 g/L for EFC+using stock glucose (360 g/L). Supplemented media were used to culture ExpiCHO (6C9) and CHO K1 cells for 14 days in a fed-batch culture. EFC+was added at 3% daily by volume on days 3-13. Cell viability, viable cell density (VCD), IgG production (titer), and specific productivity (titer/iVCD) were analyzed.

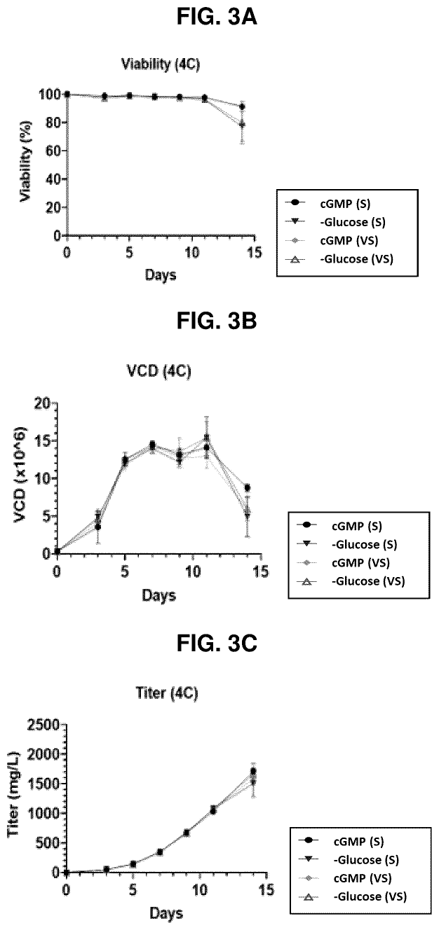

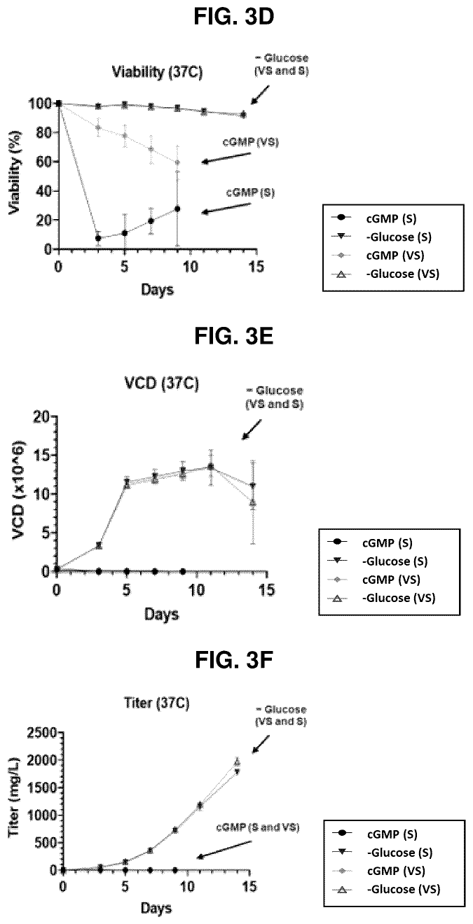

FIG. 3A-F show viability (FIG. 3A, FIG. 3D), viable cell density (VCD; FIG. 3B, FIG. 3E), and IgG titer (FIG. 3C, FIG. 3F) of ExpiCHO S (6C9) cells in DYNAMIS medium with EFC+ at TO (4° C.) (FIG. 3A-C) and after 95 days at 37° C. (FIG. 3D-F).

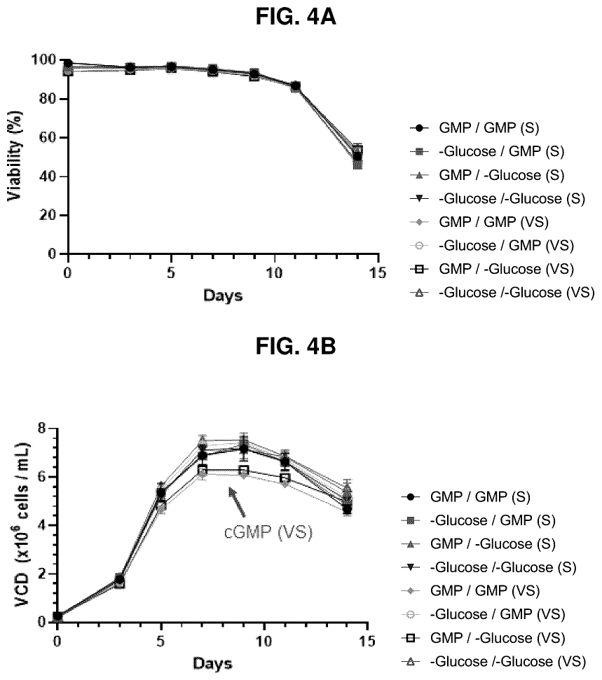

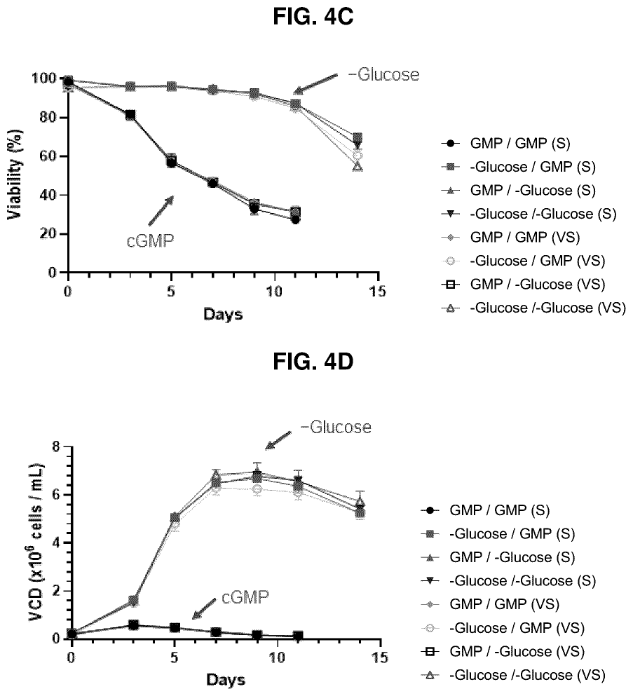

FIG. 4A-D show viability (FIG. 4A, FIG. 4C) and viable cell density (VCD; FIG. 4B, FIG. 4D) of ExpiCHO S (6C9) cells in DYNAMIS medium with EFC+after storage for 6-months at 4° C. (FIG. 4A-B) and 25° C. (FIG. 4C-D).

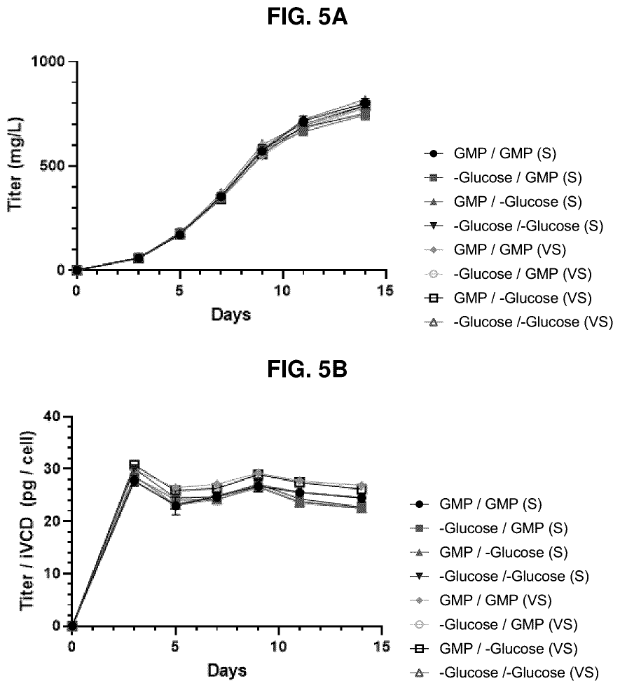

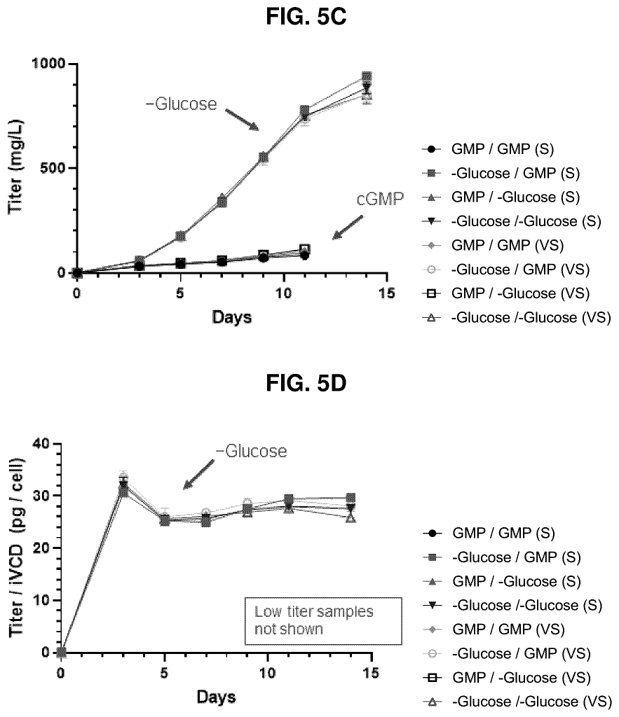

FIG. 5A-D show IgG production (FIG. 5A, FIG. 5C) and specific production (FIG. 5B, FIG. 5D) of ExpiCHO S (6C9) cells in DYNAMIS medium with EFC+after storage for 6-months at 4° C. (FIG. 5A-B) and 25° C. (FIG. 5C-D).

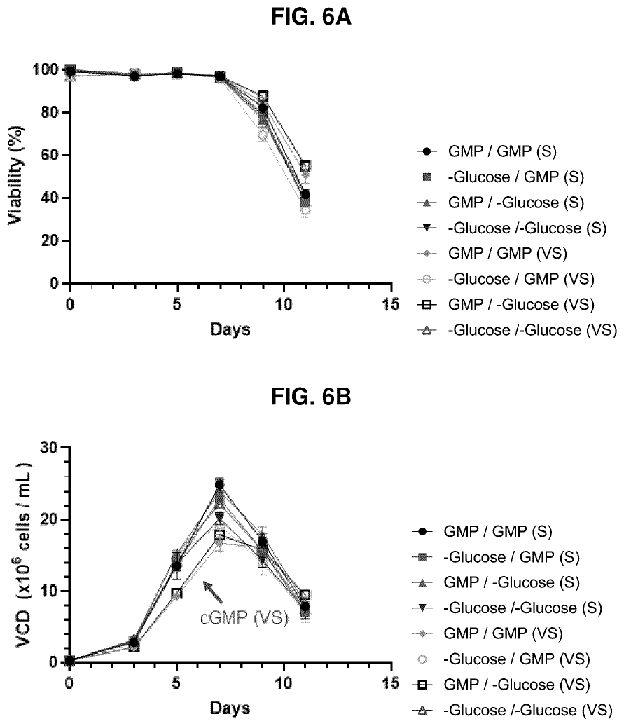

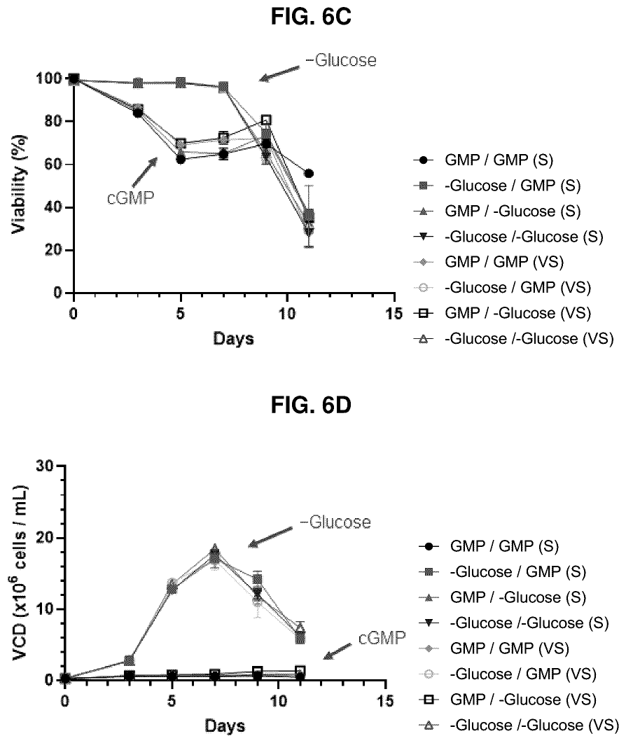

FIG. 6A-D show viability (FIG. 6A, FIG. 6C) and viable cell density (VCD; FIG. 6B, FIG. 6D) of CHO K1 (HER) cells in DYNAMIS medium with EFC+after storage for 6-months at 4° C. (FIG. 6A-B) or 25° C. (FIG. 6C-D).

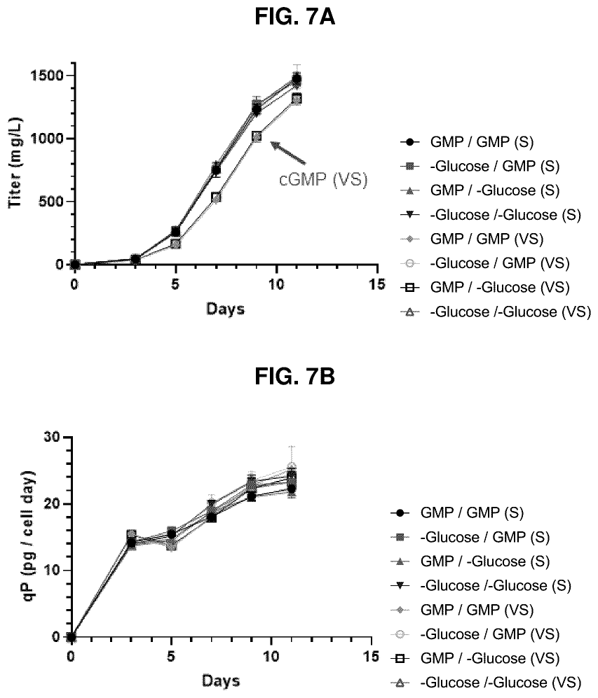

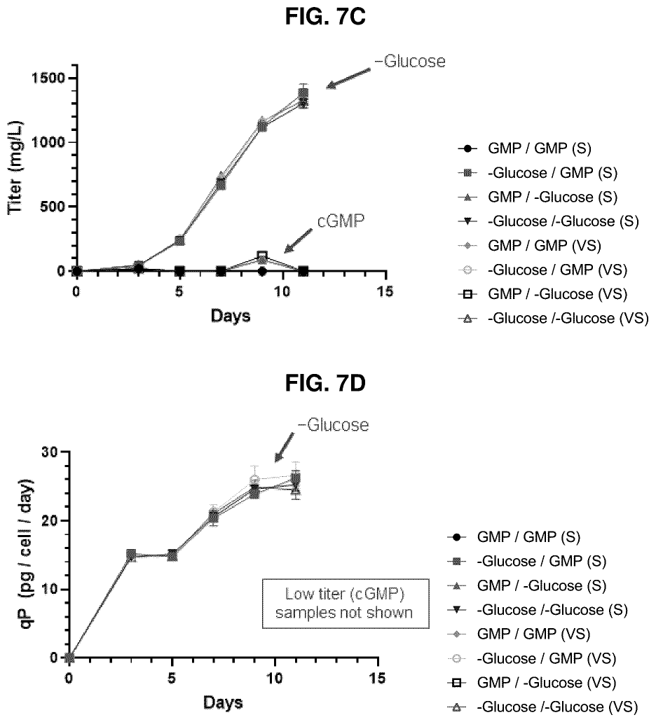

FIG. 7A-D show protein product production (FIG. 7A, FIG. 7C) and specific production (FIG. 7B, FIG. 7D) of CHO K1 (HER) cells in DYNAMIS medium with EFC+after storage for 6-months at 4° C. (FIG. 7A-B) or 25° C. (FIG. 7C-D).

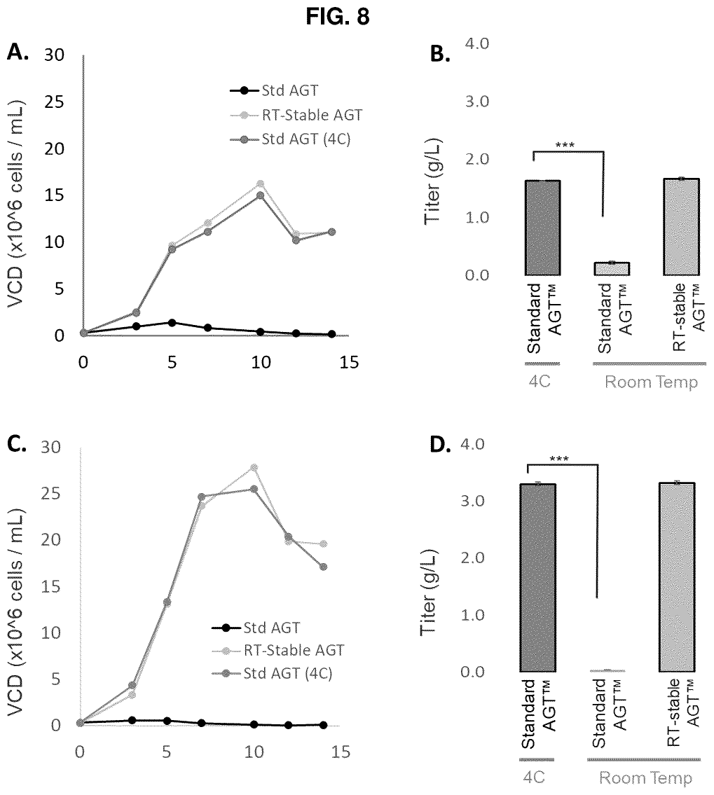

FIG. 8A-D show VCD (FIG. 8A, C) and IgG production (titer, FIG. 8B, D) of ExpiCHO S (FIG. 8A, B) and CHO K1 (HER) cells (FIG. 8C, D) cultured in DYNAMIS medium with EFC+after storage for 13 months at 4° C. or ambient (20-25° C.). CHO cells cultured in RT-stable AGT performed similarly to cells cultured in Standard (GMP) AGT that is stored at 4° C. However, cells cultured in Standard AGT that was stored at ambient temperatures (20-25° C.) for 13 months exhibited low growth (VCD) and virtually no production (titer). These data suggest that RT-stable formulations can be stored at ambient temperatures for up to 13-months with no significant deviations in performance compared to complete medium stored at 4° C.

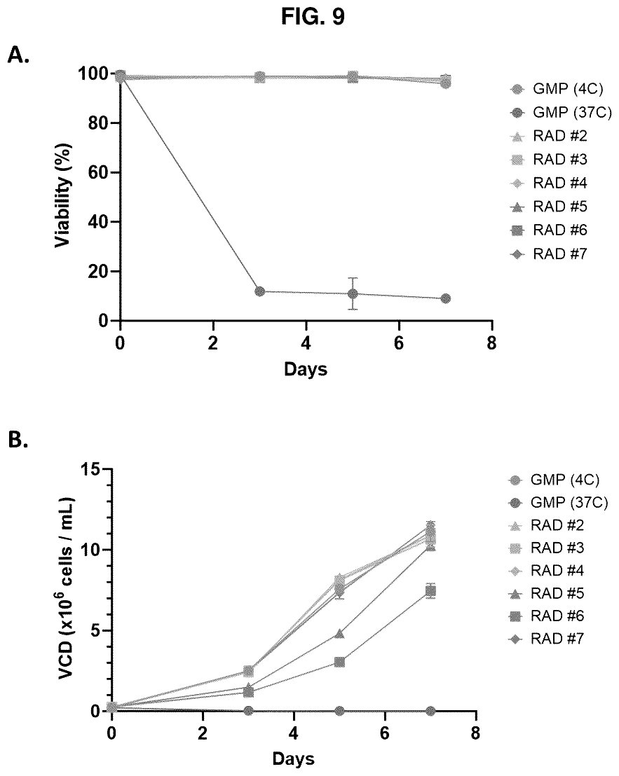

FIG. 9A, B show viability (FIG. 9A) and VCD (FIG. 9B) of a human embryonic kidney (HEK) cell line (1F11) cultured in either GMP Dynamis AGT stored at 4° C. or 37° C. for 95 days and various RT-stable (RAD #2-7) Dynamis AGT formulations stored at 37° C. for 95 days. The compositions of each of the RAD formulations (2-7) are as described in FIG. 1A-F, respectively. These data show that, like CHO cells, HEK cells exhibit poor growth in GMP medium that is stored at 37° compared to when they are cultured in GMP medium stored at 4° C. Interestingly, all of the RAD formulations (RAD #2-7) exhibit superior HEK cell growth compared to GMP Dynamis when stored at 37° C.

In all cases, it was found that CHO cells (ExpiCHO S and CHO K1) and human embryonic kidney (HEK, 1F11) cells grown in AGT medium that was separated from glucose prior to being stored at ambient or 37° C. performed significantly better (cell growth and/or IgG production) than cells grown in glucose-containing GMP AGT medium stored at ambient or 37° C. Furthermore, cells grown in-glucose AGT (or RAD formulations) that were stored at RT or 37° C. performed similarly to cells grown in medium that was stored at 4° C. These data suggest that separation of glucose from cell culture medium, prior to storage, greatly enhances the performance of multiple mammalian cell types (CHO and human cell lines) cultured in that medium when stored at RT for up to 13 months.

1. A room temperature-stable cell culture media or supplement composition comprising:
a base cell culture medium comprising one or more amino acids, salts, buffers, trace minerals, lipids, nucleic acids, or proteins;
one or more carbohydrates; and
one or more vitamins;
wherein the carbohydrates and vitamins are segregated from the base cell culture medium.

2. The composition of claim 1, wherein the composition is stable at a temperature from about 15° C. to about 30° C.

3. The composition of claim 1, wherein the composition is stable at room temperature for about 1 day to about 2 years.

4. The composition of claim 1, wherein the segregation comprises one or more of:
physical separation of the base cell culture medium from the carbohydrates and/or vitamins in different containers;
separation of the base cell culture medium from the carbohydrates and/or vitamins in the same container;
granulation or agglomeration of the base cell culture medium;
granulation or agglomeration of the carbohydrates;
granulation or agglomeration of the vitamins; or
combinations thereof.

5. The composition of claim 4, wherein the carbohydrates and/or vitamins are combined with the base cell culture medium to form the temperature-stable cell culture media or supplement composition prior to use.

6. The composition of claim 4, wherein the base cell culture medium is granulated or agglomerated and situated in a first container;
the carbohydrates are granulated or agglomerated and situated in a second container; and
the vitamins are situated in a third container.

7. The composition of claim 4, wherein the base cell culture medium and the carbohydrates are separately granulated or agglomerated, admixed, and situated in a first container; and the vitamins are situated in a second container.

8. The composition of claim 4, wherein the base cell culture medium is granulated or agglomerated and situated in a first container; and
the carbohydrates and the vitamins are admixed, granulated, or agglomerated, and situated in second container.

9. The composition of claim 4, wherein the base cell culture medium is granulated or agglomerated and situated in a first container;
the carbohydrates are granulated or agglomerated, admixed with the granulated or agglomerated base cell culture medium and situated in the first container; and
the vitamins are situated in a third container.

10. The composition of any one of claim 4, wherein an interaction or a chemical reaction of the carbohydrates and/or vitamins with the base cell culture medium is inhibited or reduced because of the segregation in separate containers.

11. The composition of claim 1, wherein the carbohydrates comprise one or more of glucose, fructose, or trehalose.

12. The composition of claim 1, wherein the one or more vitamins comprise one or more of retinol (A), thiamine (B1), riboflavin (B2), niacinamide (B3), pantothenic acid (B5), pyridoxamine (B6), biotin (B7), folic acid (B9) cobalamin (B12), ascorbic acid (C), cholecalciferol (D), tocopherol (E), phylloquinone (K), choline, inositol, lipoic acid, or para-aminobenzoic acid.

13-19. (canceled)

20. The room temperature-stable cell culture media or supplement composition of claim 1, wherein the room temperature-stable cell culture media or supplement composition is a mammalian room temperature-stable cell culture media or supplement composition.

21. A method of manufacturing the room temperature-stable cell culture media or supplement composition of claim 1, the method comprising:
(a) combining the one or more amino acids, salts, buffers, trace minerals, lipids, nucleic acids, or proteins to form the base cell culture medium;
(b) granulating or agglomerating the base cell culture medium; and
(c) combining the granulated or agglomerated base cell culture medium with granulated or agglomerated carbohydrates.

22. The method of claim 21, wherein the method further comprises preparing a solution concentrate of one or more vitamins.

23. The method of claim 22, wherein the method further comprises adding the solution concentrate of one or more vitamins to the combination of the base cell culture medium and carbohydrates upon solubilization.

24. The method of claim 23, wherein steps (b)-(c) comprise:
(c) granulating or agglomerating carbohydrates and vitamins; and
(d) combining the granulated or agglomerated base cell culture medium with the granulated or agglomerated carbohydrates and vitamins.

25-27. (canceled)

28. A kit for storing a room temperature-stable cell culture media comprising:
(a) a base cell culture medium comprising one or more amino acids, salts, buffers, trace minerals, lipids, nucleic acids, or proteins, wherein the base cell culture medium is optionally granulated or agglomerated;
(b) one or more carbohydrates, wherein the carbohydrates are optionally granulated or agglomerated;
(c) one or more vitamins; wherein the vitamins are optionally granulated or agglomerated or in a solution concentrate; and
(d) one or more containers comprising drums, buckets, boxes, bags, pouches, or combinations thereof.

29. The kit of claim 28, wherein the room temperature-stable cell culture media is used for culturing a mammalian cell in vitro.

30. The kit of claim 29, wherein the mammalian cell is a CHO cell or a HEK cell.
